SEQUENCE LISTING (17 sequences)

SEQ ID NO: 1 - protein, 9 aa, unknown
  misc_feature (1)..(9): chemically synthesized
YPYDVPDYA

SEQ ID NO: 2 - protein, 10 aa, unknown
  misc_feature (1)..(10): chemically synthesized
EQKLISEEDL

SEQ ID NO: 3 - protein, 7 aa, unknown
  misc_feature (1)..(7): chemically synthesized
DYKDDDK

SEQ ID NO: 4 - protein, 6 aa, unknown
  misc_feature (1)..(6): chemically synthesized
HHHHHH

SEQ ID NO: 5 - protein, 6 aa, unknown
  misc_feature (1)..(6): chemically synthesized
DTYRYI

SEQ ID NO: 6 - protein, 6 aa, unknown
  misc_feature (1)..(6): chemically synthesized
TDFYLK

SEQ ID NO: 7 - protein, 5 aa, unknown
  misc_feature (1)..(5): chemically synthesized
RYIRS

SEQ ID NO: 8 - protein, 6 aa, unknown
  misc_feature (1)..(6): chemically synthesized
QYPALT

SEQ ID NO: 9 - protein, 6 aa, unknown
  misc_feature (1)..(6): chemically synthesized
HTTPHH

SEQ ID NO: 10 - protein, 15 aa, unknown
  misc_feature (1)..(15): chemically synthesized
KETAAAKFERQHMDS

SEQ ID NO: 11 - protein, 12 aa, unknown
  misc_feature (1)..(12): chemically synthesized
EDQVDPRLTDGK

SEQ ID NO: 12 - protein, 6 aa, unknown
  misc_feature (1)..(6): chemically synthesized
EYMPME

SEQ ID NO: 13 - protein, 6 aa, unknown
  misc_feature (1)..(6): chemically synthesized
EFMPME

SEQ ID NO: 14 - protein, 6 aa, unknown
  misc_feature (1)..(6): chemically synthesized
PPEPET

SEQ ID NO: 15 - protein, 6 aa, unknown
  misc_feature (1)..(6): chemically synthesized
MNRLGK

SEQ ID NO: 16 - protein, 11 aa, unknown
  misc_feature (1)..(11): chemically synthesized
MASMTGGQQMG

SEQ ID NO: 17 - protein, 11 aa, unknown
  misc_feature (1)..(11): chemically synthesized
QPELAPEDPED

(19) United States
(12) Patent Application Publication
(10) Pub. No.: US 2011/0319985 A1
(43) Pub. Date: Dec. 29, 2011

(54) METHODS AND APPARATUS FOR LOCALIZED ADMINISTRATION OF INHIBITORY MOIETIES TO A PATIENT

(75) Inventor: Randolf Von Oepen, Los Altos Hills, CA (US)

(73) Assignee: ABBOTT LABORATORIES VASCULAR ENTERPRISES LIMITED, Dublin (IE)

(21) Appl. No.: 13/113,793

(22) Filed: May 23, 2011

Related U.S. Application Data
(62) Division of application No. 10/015,274, filed on Dec. 11, 2001, now abandoned.
Application No. 10/015,274 is a continuation-in-part of application No. 09/303,849, filed on May 3, 1999, now abandoned.
Application No. 09/303,849 is a continuation-in-part of application No. 09/442,591, filed on Nov. 17, 1999, now abandoned.

(30) Foreign Application Priority Data
May 5, 1998 (DE) 19819635.0
Nov. 17, 1998 (DE) 19853067.6

(51) Int. Cl. A61F 2/06

(52) U.S. Cl. 623/1.42

(57) ABSTRACT

Methods and devices are provided for the localized administration to a patient of moieties effective for inhibiting unwanted cellular growth, including restenosis of an artery treated with a stent implant for blockage of blood flow by an atherosclerotic lesion. After implantation of a medical device capable of moiety-binding, moieties effective at inhibiting unwanted cellular growth are administered locally to a patient. In this manner the deleterious side-effects of systemic administration of moieties are avoided. Upon localized administration, the moieties bind the medical device, rendering the medical device itself capable of inhibiting unwanted cellular growth. According to an embodiment, after implantation of a stent, radioactive moieties specific for receptors immobilized on the stent surface are locally administered using a balloon perfusion catheter. The moieties bind specifically the receptors, becoming immobilized thereto, thereby rendering the stent radioactive and effective for inhibiting restenosis.

### CROSS-REFERENCE TO RELATED APPLICATIONS

This application claims priority from the copending U.S. patent application Ser. No. 09/303,849, filed May 3, 1999, and Ser. No. 09/442,591, filed Nov. 17, 1999. This application also claims priority from the German patent applications No. 19819635.0, filed May 5, 1998, and No. 19853067.6, filed Nov. 17, 1998. The disclosure of each of the afore-referenced priority patent applications is incorporated by reference herein in its entirety.

### FIELD OF THE INVENTION

The present invention relates generally to the inhibition of unwanted cellular growth in the vicinity of medical devices implanted in a patient. More particularly, the present invention relates to medical devices capable of being made radioactive, or otherwise capable of inhibiting unwanted cellular growth, and methods of making such medical devices radioactive or otherwise growth inhibitory. Yet more particularly, the present invention relates to stents capable of being made radioactive, or otherwise growth inhibitory, so as to inhibit restenosis, as well as methods of making such stents radioactive or growth inhibitory through the localized administration of radioactive or other growth inhibitory moieties.

### BACKGROUND OF THE INVENTION

Over the entire course of the 20th century, cardiovascular disease (CVD) has been the number one cause of death in the United States, except for 1918, the year of the influenza pandemic. According to the American Heart Association, nearly 950,000 Americans died of cardiovascular disease (CVD) in 1998. About 48% of these deaths were attributable to coronary heart disease, or heart attack, and about 17% were attributable to cerebrovascular disease, or stroke. Importantly, nearly three-fourths of all deaths from CVD, including both heart attack and stroke, share a common etiology, atherosclerosis.

Atherosclerosis is a disease process affecting the arteries and related blood vessels. The structure of healthy arteries comprises three concentric layers: the intima, next to the lumen of the artery, and then, moving outward, the media, and finally the aventitia. The intima comprises a single layer of endothelial cells, and the media comprises smooth muscle cells with varying amounts of elastic fibers.

Atherosclerosis involves the formation of lesions, called plaques, or athermomas, that begin as a thickening of the intima of an artery, and as the disease progresses, advance into the lumen. Continued growth and development of a plaque can result in life threatening complications.

A well-accepted theory regarding the pathogenic mechanism of atherosclerosis posits that an injury to the endothelial cell lining of the intima triggers a chronic inflammatory response of the arterial wall. Injury to the endothelial cell lining may occur as a result of physical injury (i.e. denuding injury), exposure to environmental toxins (e.g. cigarette smoke), infectious agents, high cholesterol and lipid levels in the blood, and may also result from normal hydrodynamic stresses due to the flow of blood through the vessel.

In response to the injury, blood monocytes adhere to the endothelial cells, migrate between the cells and take up residence below the endothelial layer, where they differentiate into macrophages and proliferate. Smooth muscle cells migrate into the intima from the media where they too proliferate. Both macrophages and smooth muscle cells form foam cells by taking-up and accumulating lipids and lipoproteins able penetrate into the arterial wall as a result of the injury, and the smooth muscle cells produce extracellular matrix components that accumulate in the intima. These events result in the formation of a plaque that penetrates into the lumen, typically comprising a core of fatty foam cells surrounded by smooth muscle cells, and covered at the intimal aspect by the connective tissue components produced by the smooth muscle cells.

As the plaque grows into the lumen, it causes stenosis, the reduction, or even complete blockage, of the lumen of the blood vessel in which it is situated, with the consequential reduction of blood flow past it. As a result, the organ fed by that artery suffers ischemia, a physiologic state characterized by inadequate oxygen and nutrient supplies to meet the metabolic need, as well as diminished removal of metabolic waste products. At minimum, ischemia compromises the function of the organ, and if severe enough, can result in tissue and organ death. Stenosis of the coronary arteries results in ischemia of the heart tissue which can precipitate myocardial infarction (heart attack), the death of those parts of the heart muscle dependent on the particular coronary artery that is blocked.

Particularly dangerous for the patient with atherosclerosis of the coronary arteries is the occurrence of an acute plaque change. For a variety of reasons, including hemodynamic stress, and changes wrought by the cellular components of the plaque, a plaque lesion undergoes a structural change the triggers the formation of a thrombus, or blood clot.

Thrombus formation can occur either when the plaque ruptures, or fissures form in the plaque, thus exposing the internal constituents of the plaque to the blood, or when parts of the plaque erode or ulcerate, thus exposing the subendothelial basement membrane to the blood. In both cases the blood is exposed to highly thrombogenic substances in or near the plaque, causing platelet adhesion, aggregation, activation and release of molecules that further promote aggregation, resulting a rapid feed-forward cycle causing a clot to form over the disrupted plaque. At best, healing of the thrombus and disrupted plaque results in growth of the overall plaque lesion; at worst, the thrombus completely occludes the artery. In the coronary artery this often results in myocardial infarction, but can also cause sudden cardiac death if the ischemia triggers ventricular fibrillation. In the brain, blockage causes stroke. The thrombus can also embolize, throwing off small fragments that are carried downstream in the blood to lodge in smaller arteries, which can also result in heart attack and stroke.

Given the profound morbidity and mortality associated with cardiovascular disease in the United States, and in other nations that share a westernized life style, substantial effort has been invested by the research and clinical communities to understand the etiology of atherosclerosis and to develop medical and surgical interventions.

One of the most effective surgical interventions is the minimally invasive technique called percutaneous transluminal angioplasty (PTA), colloquially known as balloon angioplasty. In PTA, a physician threads the end of a specially designed catheter through the narrowed part of the artery where the atherosclerotic plaque is situated. Then, a small balloon integral with the end of the catheter is inflated with pressurized fluid. As the balloon expands it makes contact with the plaque surface and walls of the artery, and as the pressure continues to build, the plaque material is compressed against artery wall below it. In this manner, the lumen diameter is increased. After the balloon is deflated, and the catheter removed from the patient, a greater volume of blood can again flow through the artery. When the technique is used to treat stenotic coronary arteries, it is called percutaneous transluminal coronary angioplasty (PTCA).

In the first years PTCA was practiced, acute restenosis, an unwelcome complication of PTA, arose in some patients. Within hours or days of the procedure, the artery lumen opened by the balloon narrowed, again hindering blood flow. This restenosis is variably caused by elastic recoil of the vessel wall due to its being overstretched by balloon inflation (i.e. barotrauma), separation of the compressed plaque from the artery wall such that it enters and blocks the lumen, or by thrombus formation. This life threatening complication required repetition of the PTCA procedure or resort to the traditional coronary artery bypass graft procedure (CABG) to regain perfusion.

An important advance that reduced the incidence of acute restenosis, and the effectiveness of PTA generally, was the concomitant implantation of a stent as the balloon is inflated to compress the plaque lesion.

Stents are expandable mesh-like tubes, typically fabricated from a biocompatible metal such as surgical stainless steel. In the modified PTA technique, an unexpanded stent is emplaced to surround the uninflated balloon on a catheter. When the physician places the stent covered balloon inside the artery and inflates it, the stent is caused to expand with the balloon. When the balloon volume is great enough, the stent contacts the plaque and artery walls at the same time the balloon compresses the lesion.

When the balloon is deflated however, the stent remains expanded and in place, such that it provides structural support to the artery and compressed lesion, thereby reducing the propensity for acute restenosis.

However, despite this advance, by monitoring the post operative status of their patients, clinicians came to realize that within six months of PTCA and stent implantation, a substantial minority of patients (10-40%) suffer long term restenosis at the site where the stent dwells in the artery. When the extent of restenosis is severe enough, it is necessary to repeat the PTCA procedure to reopen the artery, possibly with the implantation of additional stents, or alternatively to perform bypass surgery.

The mechanism of long term in-stent restenosis apparently is different from that of acute restenosis and involves a chronic inflammatory response termed neointimal hyperplasia. When the expanding balloon and/or stent contacts the walls of the artery, the artery is injured in at least two ways. First, the thin layer of endothelial cells covering the intima of the artery is stripped away, exposing underlying extracellular matrix components. The second aspect of the injury may involve barotrauma due to over stretching of the arterial walls.

Subsequent to the balloon-induced injury, platelets from the blood adhere to the area denuded of endothelial cells and release growth and chemotactic factors that induce the proliferation and migration of smooth muscle cells from the media of the artery into the intima where they continue to proliferate and deposit extracellular matrix components. In this way the thickness of the intima increases, eventually resulting in restenosis, with the effect of again reducing bloodflow. The smooth muscle cells themselves and/or other cellular components continue to elicit factors that promote the inflammatory response such that cellular proliferation continues for up to six months after the balloon-induced injury.

Clinicians and researchers are actively pursuing strategies for inhibiting or reversing the process of neointimal proliferation before it causes restenosis so that it will be unnecessary to repeat the angioplasty and stenting. Some approaches involve administration of drugs that block one or more stages of the feed-forward cycle that results in intimal thickening. Examples include drugs that prevent platelet adhesion, aggregation, and thrombus formation, and drugs that block the stimulatory effect of cytokines and other factors on smooth muscle cell migration and proliferation.

Another approach is based on gene therapy, whereby growth inhibitory genes are introduced into smooth muscle cells in the vicinity of the implanted stent such that expression of the introduced genes will tend to reduce the degree of intimal proliferation. One means by which to introduce the genes is by infection of smooth muscle cells by recombinant viruses, such as adenovirus. Another means, as disclosed in U.S. Pat. No. 6,129,705, is to coat the exterior of an angioplasty balloon or stent with microspheres containing a gene therapy agent. As the balloon or stent is expanded the microspheres contact an artery wall, and after sufficient pressure is exerted, rupture, such that the cells of the artery wall are exposed to the agent.

In yet another approach to controlling restenosis, clinicians and researchers are employing ionizing radiation to inhibit neointimal proliferation. This approach is known as brachytherapy, and relies on the now well known phenomenon that when dividing cells are exposed to sufficiently high does of ionizing radiation, for example, in the form of beta particles, alpha particles or x-ray or gamma radiation, DNA strand breaks disrupt the normal process of cell division, or mitosis, thereby inhibiting or even stopping cellular growth. This effect of ionizing radiation on cell growth underlies its use as a cancer therapy modality, and is also being explored as a means to control restenosis following PCTA.

Different means for brachytherapy have been designed and are being studied for safety and efficacy in large-scale clinical trials. In one approach, a radioactive source is contained inside a catheter's distal end. Sources being tested include beta particle and gamma radiation emitting radionuclides. Most sources are solids, but in at least one system a liquid is used. This brachytherapy approach entails advancing the catheter into a coronary artery previously treated by PTCA until the radiation source is close to the compressed plaque lesion. The source is kept in place until such time that the lesion and adjacent healthy artery have absorbed a predetermined optimal dose of radiation, after which the catheter is removed.

Although evidence suggests that catheter-based brachytherapy may be effective, this approach presents problems. One limitation involves the tradeoff between the time necessary for the optimal dose to be absorbed and the degree of radioactivity of the source. Generally, in the interest of minimizing unwanted radiation exposure to medical personnel and the patient it is desirable to minimize the radioactivity of the source. However, the lower the activity of the source, the greater the amount of time the catheter must be in-place in the patient for the required dose of radiation to be absorbed. Increased dwell-time reduces the total number of patients that a busy angioplasty team can treat, but more importantly, increases the opportunity for unwanted side effects, such as thrombosis formation, to occur.

Additionally, for the reasons just discussed, the radiation dose must be administered in a single brief session, typically just after PTCA. Thus, using the catheter-based technique, there is no ability to administer the same dose over substantially longer periods. As a consequence, because there is a lag period between PTCA and when the smooth muscle cells begin to proliferate, they are not exposed to radiation when they most radiosensitive, i.e., during mitosis.

As an alternative to catheter-mediated brachytherapy, a number of investigators have developed means by which to make the stent itself radioactive, such that upon implantation, radiation emitted by the stent has been demonstrated in clinical trials to inhibit smooth muscle cell growth and restenosis. An advantage of this approach is that the radiation dose can be administered over longer time periods than is possible with catheter-based techniques, and mitotic cells are exposed to radioactivity.

For example, U.S. Pat. No. 6,077,413 discloses methods and apparatus relating to the deposition of a radioactive metal layer onto the stent, such as by electroplating. U.S. Pat. No. 6,159,142 also discloses coating a stent, but with a biodegradable coating comprising a radionuclide, such that the radioactivity has decayed by the time the coating is degraded. U.S. Pat. No. 6,152,869 discloses surrounding a stent with a polymeric sleeve comprising radionuclides, and U.S. Pat. No. 6,192,095 discloses making a stent radioactive by ion injecting radioactive xenon ions into the stent surface.

A common limitation of all these approaches whereby the stent is made radioactive first and subsequently implanted into a patient is that it is not generally possible to prepare the radioactive stent for implantation in the hospital.

Typically, special equipment and expertise is necessary to load a stent onto the balloon catheter used to deploy the stent. Thus, regardless of the isotope used, the process of making a stent radioactive must be performed at the time of manufacture of the stent or at the time the apparatus comprising the stent and balloon catheter is assembled. This raises logistical and safety problems associated with shielding the radioactive stent assembly during distribution to the end user. Logistical problems are exacerbated when the radioactive stent comprises a short-half life radionuclide, such as ³²P (t_{1/2}=14.3 days), because reliable availability of a stent with a predefined radiation dose would require a well coordinated manufacturing and distribution system to ensure that the stent reaches the end user before the radioactivity decays to ineffective levels. This would make radioactive stents much more expensive to the end user.

A novel and surprising approach for avoiding the problems associated with making and using radioactive stents, as discussed above, is to make a non-radioactive, or “cold” stent, radioactive after such time that the stent has been implanted in the patient's artery.

A means for making a stent radioactive after implanting a non-radioactive stent in a patient is disclosed in U.S. Pat. No. 5,925,353. The invention provides a stent to which is attached antigen molecules. After the stent is implanted, radioactively labeled antibodies are administered, e.g., intravenously, to the patient, whereby the antibodies bind the antigen on the stent, making it radioactive.

A similar invention is disclosed in International Application PCT/US98/23022. This invention provides a stent having a first substance immobilized on the stent surface, which substance has a strong and selective affinity for binding a second, radioactive substance. The nonradioactive stent, bearing only the first substance, is first implanted. Subsequently, the radioactive second substance is introduced in liquid form into the blood stream of the patient. As the radioactive substance circulates throughout the vascular system of the patient, it eventually passes through the artery where the stent is implanted. Upon contact, the first and second substances bind each other, and as the second radioactive substance thereby becomes immobilized on the stent, the stent is made increasingly radioactive. When the first substance is saturated, or when all the second substance has been bound, the stent is rendered maximally radioactive.

The inventions described above suffer several substantial drawbacks however. Most importantly, both entail systemic administration (e.g. intravenously) of a radioactive substance to a human patient. As a result, the entire vascular system, and all organs and tissues are exposed to the radioactive substance. At minimum, this results in unnecessary full-body exposure of the patient to radioactivity. In fact, the entire dose of radioactivity remains in the body until excreted in urine and/or feces.

Also, as a consequence of systemic administration, the radioactive substance becomes highly diluted in the volume of circulating blood. Further, the circulating concentration does not remain constant, but declines as the substance is metabolized, principally by the kidneys and liver. Lastly, the radioactive substance may be preferentially absorbed and sequestered in particular compartments, such that binding to nonspecific sites within the body competes for binding to the specific sites (i.e., immobilized first substance). For example, if the radioactive substance is partially lipophilic, it may be sequestered in adipose tissue.

To compensate for these effects, and to ensure that an effective amount of the radioactive substance becomes immobilized on the stent, it is either necessary to choose the first and second substances (i.e., binding pair) such that their binding affinities are extremely high, or it is necessary to administer a large amount of radioactive substance to ensure that its concentration in the blood is sufficiently high to ensure binding.

In the first instance, selection of binding pairs is necessarily limited to only those exhibiting highest affinity binding. If the affinity is high enough, the binding is effectively irreversible, making it practically impossible to remove the radioactive substance, should that be desired.

In the second instance, there exists the risk of potentially toxic effects from introducing a large amount of a foreign substance into the body. Administration of a larger amount of the radioactive substance, of course, also increases the whole body exposure. To minimize the full body exposure, it may be necessary to choose a radioactive nuclide that emits radiation at lower energies. However, this has the consequence of reducing the efficacy of the stent once it has become radioactive.

Systemic administration of large quantities of a radioactive substance also suffers from the problem that, as the substance is metabolized and excreted, the urine and feces become contaminated with radioactivity, necessitating expensive and inconvenient collection and disposal.

In light of the aforementioned disadvantages associated with implanting a radioactive stent, and of systemic administration of a radioactive substance so as to make a stent radioactive after implantation, there remains a need in the art for methods by which a nonradioactive stent, having been implanted in a patient's artery, may be made radioactive without introducing substantial amounts of a radioactive substance into the systemic blood volume.

### SUMMARY OF THE INVENTION

In view of the foregoing, it is an object of the present invention to provide methods of making medical devices implanted in a patient capable of inhibiting unwanted cellular growth, but in a manner that obviates the disadvantages of implanting a medical device that is already radioactive, or the disadvantages of systemic administration to the patient of radioactive moieties and other moieties capable of inhibiting unwanted cellular growth.

It is, therefore, a further object of the present invention to provide methods of localized administration to a patient of such radioactive and other moieties for the purpose of rendering an implanted medical device capable of inhibiting unwanted cellular growth.

It is yet a further object of the present invention to provide medical devices that can be rendered capable of inhibiting unwanted cellular growth after implantation in a patient by the methods of localized administration provided herein.

It is yet another object of the present invention to provide methods for inhibiting restenosis after stent implantation by localized administration to a patient of moieties capable of inhibiting restenosis, including radioactive moieties, neutron-capture moieties, and other moieties capable of inhibiting restenosis or unwanted cellular growth.

It is a further object of the present invention to provide stents that can be rendered capable of inhibiting restenosis after implantation in a patient by the methods of localized administration of radioactive moieties and other moieties capable of inhibiting restenosis.

Thus, in a first aspect, the invention provides a method of rendering a medical device, already implanted in a patient, capable of inhibiting unwanted cellular growth, comprising administering locally to a patient a moiety capable of inhibiting unwanted cellular growth, wherein the medical device binds the moiety.

According to a second aspect, the invention provides a method of treating a patient, implanted with a stent, and in need of therapy for restenosis, comprising administering locally to said patient a moiety capable of inhibiting restenosis, wherein the stent binds the moiety.

In a third aspect, the invention provides a method of making a stent, implanted in a patient, radioactive, comprising administering locally to a patient a radioactive moiety comprising a second member of a specific binding pair, wherein the stent comprises a first member of the specific binding pair capable of specific binding with the second member.

In a fourth aspect, the invention provides a method of making a stent, implanted in a patient, radioactive, comprising administering locally to a patient a neutron-capture moiety comprising a second member of a specific binding pair, and exposing the stent to a neutron flux, wherein the stent comprises a first member of the specific binding pair capable of specific binding with the second member.

In a fifth aspect, the invention provides a method of making a stent, implanted in a patient, capable of inhibiting restenosis, comprising administering locally to a patient a restenosis inhibitory moiety comprising a second member of a specific binding pair, wherein the stent comprises a first member of the specific binding pair capable of specific binding with the second member.

According to particular embodiments, the moiety that is administered locally comprises a second member of a specific binding pair, and the medical device comprises a first member of the specific binding pair capable of specific binding with the second member.

In other embodiments, the first and/or second members of the specific binding pair can comprise biomolecules. The first member can be immobilized directly to the medical device, or can be immobilized to a coating thereof, which coating can comprise a polymer or a ceramic.

In yet other embodiments, the moiety comprises a radioactive atom, and is thereby rendered radioactive. According to other embodiments, the moiety comprises a neutron-capture moiety and can be made radioactive by exposure to a neutron flux.

In yet further embodiments, the moiety comprises a restenosis inhibitory moiety that can comprise a growth inhibitory moiety, or alternatively, a restenosis inhibitory moiety.

The methods of the present invention in their various embodiments can comprise the further step of repeating, at least once, the step of administering locally to a patient a moiety capable of inhibiting unwanted cellular growth, or alternatively, a moiety capable of inhibiting restenosis. In all such steps of local administration to a patient, a balloon delivery catheter can usefully be employed.

The invention also provides for medical devices, including stents, rendered capable of inhibiting unwanted cellular growth by the methods disclosed herein. Yet further embodiments provide for stents made radioactive by the methods disclosed herein.

According to yet further embodiments, kits are provided for rendering a medical device capable of inhibiting unwanted cellular growth including a moiety capable of inhibiting unwanted cellular growth and instructions effective for performing the methods of the invention. Such kits can also include a medical device, including a stent, in addition to a means for localized administration of the moiety to a patient.

Kits are also provided for making stents radioactive, including a radioactive moiety, or alternatively a neutron-capture moiety, and instructions effective for performing the methods of the invention. Such kits can also include a stent, in addition to means for localized administration of the radioactive, or neutron-capture moiety to a patient.

Kits are also provided for making a stent capable of inhibiting restenosis, including a restenosis inhibitory agent, and instructions effective for performing the methods of the invention. Such kits can also include a stent, in addition to means for localized administration of the restenosis inhibitory moiety to a patient.

### BRIEF DESCRIPTION OF THE DRAWINGS

FIG. 1 is an orthogonal sectional view down the axis of an artery in which is implanted an embodiment of the stent of the present invention for preventing restenosis of the artery. The top part of FIG. 1 shows a full sectional view of the artery and stent. The bottom part of FIG. 1 shows an enlarged partial sectional detail of the artery and stent, including an embodiment of the stent of the present invention wherein radioactive moieties are immobilized on the stent.

FIG. 2 is similar to FIG. 1 and shows an alternative embodiment of the stent of the present invention for preventing restenosis of the artery wherein neutron-capture moieties are immobilized on the stent.

FIG. 3 is similar to FIG. 1 and shows an alternative embodiment of the stent of the present invention for preventing restenosis of the artery wherein restenosis inhibitory moieties are immobilized on the stent.

FIG. 4 illustrates an embodiment of the method of the present invention of local administration to a patient of moieties effective for inhibiting restenosis of an artery. The figure shows an elevation view, partly in section, of an artery in which is implanted a stent of the present invention, and steps for the localized administration of restenosis-inhibiting moieties.

FIG. 5A and FIG. 5B illustrate an alternative embodiment of the method of the present invention of local administration to a patient of moieties effective for inhibiting restenosis of an artery.

### DETAILED DESCRIPTION OF THE INVENTION

The present invention provides for medical devices capable of being made radioactive, or otherwise capable of inhibiting unwanted cellular growth, including restenosis, after implantation into a patient. The present invention also provides for methods of making such medical devices radioactive, or otherwise capable of inhibiting unwanted cellular growth, including restenosis, after implantation into a patient.

According to the present invention, numerous types of medical devices may usefully be made radioactive, or otherwise capable of inhibiting unwanted cellular growth. Examples include but are not limited to stents, coils, shunts, pins, plates, meshes, particles, spheres, vascular grafts, artificial valves, artificial joints, cannulas, orthopedic burs, catheters, electrode leads, filters, needles, prostheses, and patches. Additional medical devices useful for purposes of the present invention are within the knowledge of the skilled artisan.

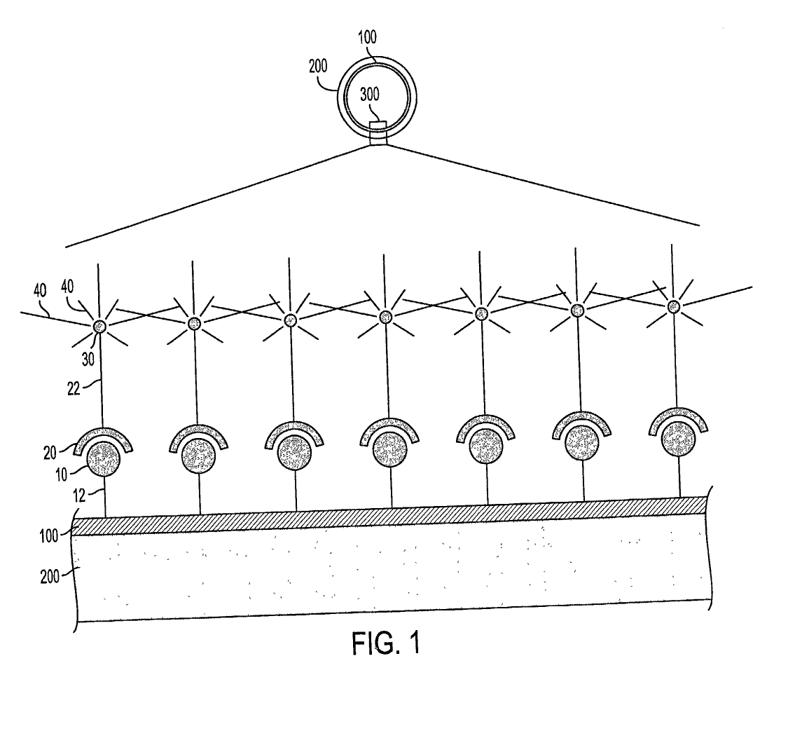

According to an embodiment of the present invention a medical device that is capable of being made radioactive, or otherwise capable of inhibiting unwanted cellular growth, is a stent. Stent 100, according to this embodiment, is schematically represented in FIG. 1. Shown in the top aspect of FIG. 1 is an axial cross section of an artery 200 in which is implanted stent 100. In the bottom aspect of FIG. 1 is shown a magnified view of a subsection 300 of the axial cross section of the artery-stent combination. As shown, stent 100 contacts the wall of artery 200, and faces the lumen of the artery. Contacting or attached to stent 100 through a linker 12 are a plurality of a first member 10 of a specific binding pair. The second member 20 of the specific binding pair is shown non-covalently bound to the first member 10. Additionally, second member 20 is attached through linker 22 to radioactive moiety 30 that emits radiation 40 through radioactive decay, which radiation contacts the cells comprising the artery walls.

Although not evident from the view shown in FIG. 1, when implanted and in contact with the wall of an artery, most stents according to the instant invention allow for communication between the artery lumen and a substantial proportion of the surface area of the artery wall. For example, as appreciated by the skilled artisan many stent designs provide for numerous, wide-open fenestrations when the stent is in its expanded state. In this manner, radiation 40 is not substantially absorbed by the stent and is instead available for absorption by cells of the artery.

Stent 100 may be of the self-expansible type, or of the type that requires the application of radial force to expand it against the artery wall and fix it in place.

Stent 100 is manufactured from materials and according to techniques well known in the art. As understood by the skilled artisan different materials are useful for the manufacture of stent 100, including but not limited to plastics, other polymeric materials, ceramics and metals, such as surgical stainless steel, tantalum, platinum, titanium, tungsten alloy, or nickel-titanium shape-memory alloy. Desirable qualities possessed by the material include but are not limited to flexibility, ductility, malleability, deformability, biocompatibility, resistance to corrosion, and radio-opacity. Materials of which stent 100 are manufactured may be amorphous, metallic or substantially crystalline in atomic or molecular structure, although other structures will be within the knowledge of the skilled artisan.

Stent 100 is typically fabricated from a tube or sheet rolled into a tube-like form. To create an expandable mesh-like structure useful for stent function material must be removed from the tube or sheet, such as by a commercially available computer-controlled laser, chemical etching using photoresistive masking techniques, machining, electrode discharge machining (EDM), or cutting with a water jet. Stent 100 may also be constructed by welding, bonding, gluing or otherwise joining discrete structural members together, or by molding or casting. Other techniques for fabricating stent 100 are known within the knowledge of the skilled artisan.

Referring again to FIG. 1 one or more first members 10 of a specific molecular binding pair are immobilized on one or more surfaces of stent 100 through linker moiety 12, and are accessible from the lumen of the artery. According to an alternative embodiment, first member 10 is not immobilized directly to a surface of the stent, but rather to a coating that covers at least a portion of the stent surface (not shown).

The configuration shown in FIG. 1 should not be construed as limiting. As will be appreciated by the skilled artisan, many other stent designs are compatible with immobilization of a first member of a specific binding pair to a surface of a stent that is accessible from the artery lumen.

Linker 12 is a molecular moiety interposed between the first member 10 of the specific binding pair, and the stent surface, such that one end contacts the member 10, and the other end contacts the stent 100. Typically, but not necessarily, linker 12 does not contribute to the specific interaction between the first 10 and second 20 members of the binding pair, but rather provides a flexible linkage between first member 10 and the stent surface, thus improving the accessability of the second member 20 to first member 10.

According to the knowledge of the skilled artisan, linker 12 may comprise a modified or unmodified biomolecule, an organic molecule, an inorganic molecule, or a combination of biomolecules, organic molecules, or inorganic molecules. Where, as is typical, a plurality of a particular species of first member 10 is to be immobilized to a stent, different types of linkers may be used to join first member 10 to a surface of stent 100 according to the knowledge and requirements of the skilled artisan.

Biomolecules useful for constructing linkers include but are not limited to proteins, peptides, and amino acids; nucleic acids, including ribonucleic acid and deoxyribonucleic acids, nucleosides and nucleotides; lipids, including phospholipids and fatty acyl chains; and complex and simple carbohydrates, including monosaccharides, disaccharides, and higher order branched and unbranched polysaccharides. Organic and inorganic molecules useful for purposes of the present invention are known to the skilled artisan.

Methods for attaching linker moiety 12 to first member 10 are within the knowledge of the skilled artisan, and include chemical and biological processes.

Additional information regarding the structure and composition of linkers and methods for joining linkers to other molecules may be found in “Bioconjugate Techniques,” G. T. Hermanson, Academic Press, Inc., San Diego, 1996, which is incorporated herein by reference in its entirety.

According to an alternative embodiment of the present invention, linker 12 is dispensed with entirely, and first member 10 of the specific binding pair is immobilized directly to the surface of stent 100, or to a coating covering at least a portion of the surface of the stent.

Hereafter, unless otherwise indicated, in discussing immobilization to a stent the phrase “first member of a specific binding pair,” or simply “first member,” refers both to a molecular entity immobilized to a stent directly, without the participation of a linker moiety, as well a molecular entity immobilized to a stent indirectly through a linker.

As mentioned above, first member 10 of the specific binding pair may be immobilized to a coating covering at least a portion of a surface of the stent. Thus, a coating may cover continuously or discontinously all surfaces, edges or component structural members of the stent; a single surface, e.g., the surface facing the arterial wall, or the surface facing the lumen of the artery; or one or more discrete portions of one or more stent surfaces, edges or component structural members. Also falling expressly within the definition of coating would be a material that fills holes or depressions formed in the material of which the stent is composed.

In all cases, the material chosen for coating should be biocompatible, inducing neither substantial toxic effects in a patient, nor a substantial immune reaction.

Coatings may usefully comprise polymers, including simple or branched homopolymers comprising a single species of monomer; simple or branched copolymers comprising two or more species of monomers; and mixtures of different types of polymers. Other materials suitable for use as coatings will be apparent to those of skill in the art and include but are not limited to ceramic coatings. Polymers useful for coating the stent of the instant invention include biodegradable polymers, as well as biostable polymers that tend to resist degradation. The choice of polymer type is dictated, in part, by the desired chemical, physical and biological properties of the resulting coating, and is made according to the knowledge and requirements of the skilled artisan.

The molecular structure of biodegradable polymers is susceptible to degradation by chemical or biological processes that occur naturally in association with a living organism, including enzymatic and cellular processes. Thus, after implantation into a patient, the coating of a stent comprising a biodegradable polymer will be gradually and continually removed wherever the coating is exposed to cells, enzymes or other degradive agents derived from the patient's body.

In contrast, a biostable polymer is substantially resistant to degradation by chemical and biological processes ongoing in a patient's body, and a coating so-comprised will tend to remain substantially intact, even over long periods.

Biodegradable coatings are useful for coating stents in circumstances where it is desirable to make the stent radioactive once, or at most a few times, with a radioisotope of relatively short half-life, according to the knowledge of the skilled artisan. The coating composition is chosen such that only after the radioactivity decays to a low level does the coating begin to be substantially removed from the stent surface by endogenous biological processes.

Examples of substantially biodegradable polymeric materials include, but are not limited the entries in the following Table 1.

TABLE 1

1,4-dioxepan-2-one homopolymers and copolymers
1,5-dioxepan-2-one homopolymers and copolymers
6,6-dimethyl-1,4-dioxan-2-one homopolymers and
copolymers
aliphatic polycarbonates
aliphatic polyesters
copoly(ether-esters) (e.g., PEO/PLA)
cyanoacrylates
glycolide (including glycolic acid) homopolymers and
copolymers
hydroxybutyrate homopolymers and copolymers
hydroxyvalerate homopolymers and copolymers
lactide homopolymers and copolymers
para-dioxanone homopolymers and copolymers
p-dioxanone and lactide copolymers
p-dioxanone and trimethylene carbonate copolymers
poly(amino acids)
poly(anhydrides)
poly(DL-lactic acid) (DL-PLA)
poly(glycolic acid-cotrimethylene carbonate)
poly(hydroxybutyrate-co-valerate)
poly(iminocarbonate)
poly(lactide-co-glycolide)
poly(L-lactic acid) (L-PLA)
polyalkylene oxalates
polyamides
polyamidoesters
polycaprolactone (PCL)
polydioxanone
polyorthoester
polyoxaesters
polyoxaesters containing amido groups
polyphosphazenes
polyphosphoester
polyphosphoester urethane
trimethylene carbonate (and its alkyl derivatives)
homopolymers and copolymers
trimethylene carbonate and glycolide copolymers
trimethylene carbonate and lactide copolymers
ε-caprolactone and glycolide copolymers
ε-caprolactone and lactide copolymers
ε-caprolactone and p-dioxanone copolymers
ε-caprolactone homopolymers and copolymers

A substantially non-biodegradable coating is chosen when long-term exposure of the artery to radioactivity is desired, or when it is foreseen that the radioactivity of the stent will be recharged multiple times, as discussed elsewhere in this specification.

Examples of substantially non-biodegradable polymeric materials include, but are not limited to the entries in the following Table 2.

TABLE 2

acrylic polymers (including methacrylate) and
copolymers
alkyd resins
cellophane
cellulose
cellulose acetate
cellulose acetate butyrate
cellulose butyrate
cellulose ethers (e.g., carboxymethyl cellulose and
hydoxyalkyl celluloses)
cellulose nitrate
cellulose propionate
epoxy resins
ethylene-alphaolefin copolymers
fluorocarbon polymers
hydrogels formed from crosslinked polyvinyl
pyrrolidinone and polyesters
Hydrolast ® (Advanced Surface Technology, Billerica,
MA)
Parylene ® (Advanced Surface Technology, Billerica,
MA)
Parylast ® (Advanced Surface Technology, Billerica,
MA)
poly(meth)acrylates
poly-2-hydroxylethylmethacrylates
polyacrylamido-2-methyl-1-propanesulfonic acid
polyacrylic acid
polyacrylic amides
polyacrylonitrile
polyalkyl oxides (polyethylene oxide)
polyalkylacrylates
polyalkylmethacrylates
polyamides
polyamides, e.g., Nylon 66 and polycaprolactam
polyaminoalkyl methacrylate
polycarbonates
polyester
polyesteramides
polyethers
polyethylene
polyethylene carbonates
polyethylene glycols
polyethylene oxide (PEO)
polyethylene teraphthalate
polyethylene vinyl acetate (EVA)
polyethyleneimine
polyhydroxylalkyl acrylates
polyhydroxylalkyl methacrylates
polyimides
polyiminocarbonates
polyionenes
polyisobutylene
polyisobutylene and ethylene-alphaolefin copolymers
polylysine
polymethacrylic amides
polyolefins
polyorthoesters
polyoxymethylenes
polypropylene
polystyrene sulfonic acid
polysulfonic acid
polyurethane
polyvinyl alcohols
polyvinyl aromatics, e.g., polystyrene
polyvinyl esters, e.g., polyvinyl acetate
polyvinyl ethers, e.g., polyvinyl methyl ether
polyvinyl ketones
polyvinyl pyrrolidone
polyvinyl sulfate
polyvinylidene halides, e.g., polyvinylidene fluoride
and polyvinylidene chloride
polyacrylonitrile
polyvinylpyridine
poly-β-hydroxybutyrates
rayon
rayon-triacetate
silicone polymers
vinyl halide polymers and copolymers, e.g., polyvinyl
chloride
vinyl monomer and olefin copolymers, e.g.,
etheylene-methyl methacrylate copolymers,
acrylonitrile-styrene copolymers, ABS resins and
ethylene-vinyl acetate copolymers

According to the knowledge of the skilled artisan, the polymer coating may also usefully comprise a hydrogel that absorbs water and provides a large number of free hydroxyl groups useful for immobilizing the first member of a specific binding pair.

Suitable polymeric materials for producing a hydrogel include but are not limited to the entries in the following Table 3.

TABLE 3

acrylic acid polymers and copolymers
acrylic acid ester polymers and copolymers
methacrylic acid polymers and copolymers
methacrylic acid ester polymers and copolymers
acrylonitrile polymers and copolymers
polyvinyl alcohols
glycophase
poly(2-hydroxyethylmethacrylate) (polyHEMA)
hydroxyethyl methacrylate-methyl methacrylate
copolymer

Preparation of Solutions Useful for Coating stents with polymer coatings with desired chemical and physical characteristics is within the knowledge of the skilled artisan. Typical factors to be considered in preparing such coating solutions include but are not limited to the identity of the monomeric polymer precursor molecules, cross-linking agents and solvents to be included in the coating solution; the ratio of monomers to solvent on a weight by weight or weight by volume basis; the ratio of different monomers if a copolymer coating is desired; the ratio of monomers to cross-linking agents; and conditions necessary to trigger, sustain and control the polymerization reaction.

Prior to applying the coating to the stent, it may be desirable to activate the surface of the stent to modify its physical and/or chemical properties, e.g., increase its hydrophilicity, to thereby increase the strength of interaction between the surface and any overlying polymeric coating applied thereto.

Techniques for activating the stent surface are known in the art, and include, among others, etching the surface with strong acids or bases, exposing the stent to plasma glow discharge, ozone gas, CO₂ gas plasma treatment, electron beam treatment, corona discharge, ceric ion initiation, UV light irradiation, gamma ray and X-ray treatment.

In addition to increasing the hydrophilicity of the stent surface, activation of the stent surface may be desirable to increase the density of chemical functional groups, such as metal oxides and hydroxyl groups, useful for creating covalent or other types of bonds between the stent surface and either a coating material or a first member of a specific binding pair. Chemical reactions for creating such bonds, and conditions necessary to effect such reactions are within the knowledge of the skilled artisan.

According to techniques known in the art, the unpolymerized coating solution may be applied by vapor deposition, or as a liquid, either by dipping the stent into a liquid filled reservoir, or by spraying the liquid coating material onto the surface of the stent to be coated. Other techniques known in the art for applying the coating may be used as well. After the coating is applied, polymerization is allowed to proceed, and the solvent is allowed to evaporate, leaving a solid, or semi-solid, flexible, polymeric coating adherent to, or otherwise chemically bonded to the stent surface.

A single layer of a coating may be applied, the thickness of which may be variable or constant across the stent surface as determined by the needs of the skilled artisan. Alternatively, multiple layers may be applied, the thickness of which may also be variable or constant. Each layer may have the same or a different composition.

According to an alternative embodiment of the present invention, the first member 10 of the specific binding pair is mixed with the coating solution before the coating is applied to the stent. In this manner, the first member is immobilized by being embedded in the coating once it has formed.

Depending on the nature of the coating material, embedded first members may only be accessible by soluble second members at the surface of the coating. Alternatively, if the coating material has a more open structure, it may be possible for soluble second members to diffuse into the coating such that internally embedded first members can thereby be bound.

If the coating material is biodegradable, it is expected that embedded first members would gradually and continually be released from the coating as the immobilizing matrix of the coating material dissolves or is otherwise degraded. As a result, any second members of the specific binding pair bound to immobilized first members would also be released. Thus, this embodiment is useful if it is desirable for radioactive second members to be removed from the stent without the need for further intervention. According to the knowledge and requirements of the skilled artisan, drugs and other pharmaceutically active agents can also be embedded in the coating material to be gradually and continually released as the coating material degrades in the patient's body.

Whether a first member of a specific binding pair is to be immobilized directly to the stent surface, or to an overlying coating, immobilization is effected by the creation of one or more types of chemical bonds, or a combination of such bonds, between the first member and the stent or coating. Exemplary chemical bonds include but are not limited to covalent bonds, dative-type bonds, coordinate covalent bonds, hydrophobic interactions, hydrogen bonds, ionic bonds, electrostatic (charge-charge) interactions, salt-bridge bonds and Van der Waals bonds.

Chemical bonds for effecting immobilization are created between a chemical functional group possessed by a first member of a specific binding pair, and a complementary chemical functional group either present on the stent surface, or possessed by the coating material. According to an alternative embodiment, a chemical functional group is possessed by a linker moiety attached to a first member of a specific binding pair.

Typically, a first binding pair member possesses a single species of functional group for immobilization, whereas the stent or coating material may possess a single species, or two or more different groups in proportions determined by the skilled artisan.

As understood by the skilled artisan, there is no requirement that the nature of the bond that effectively immobilizes the first binding pair member to the stent or coating be known or even presently ascertainable. There is also no requirement that the identity of the functional groups that form effectively immobilizing bonds be known or presently ascertainable. Effective immobilization can be determined empirically by the skilled artisan, without regard to the mechanism by which it occurs.

Many pairs of complementary chemical functional groups that can be combined to form bonds effective for immobilizing a first member of the specific binding pair are known to the skilled artisan, as are the reaction conditions necessary to effect the creation of a bond or bonds between chosen pairs of functional groups. A nonexhaustive list of functional groups, complementary functional groups, and/or types of reactions that may usefully be performed on functional groups to create bonds effective for immobilization are listed in the following Table 4.

TABLE 4

| | Complementary Functional |
|---|---|
| Functional Group | Group or Reaction Type |
| Amines | Isothiocyanate |
| | Isocyanate |
| | Acyl azide |
| | NHS esters |
| | Sulfonyl chlorides |
| | Aldehydes and glyoxals |
| | Epoxides and oxiranes |
| | Carbonates |
| | Arylating agents |
| | Imidoesters |
| | Carbodiimides |
| | Anhydrides |
| | Phosphate |
| | Sulphate |
| | Carboxylate |
| Thiols or Sulfhydryls | Haloacetyl and alkyl |
| | halide derivatives |
| | Maleimides |
| | Aziridines |
| | Acryloyl derivatives |
| | Arylating agents |
| | Thiol-disulfide exchange |
| | reagents, including |
| | pyridyl disulfides, TNB- |
| | thiol, disulfide |
| | reductants |
| Carboxylates | Diazoalkanes and |
| | diazoacetyl compounds |
| | Carbonyldiimidazole |
| | Carbodiimides |
| Hydroxyls | Epoxides and oxiranes |
| | Carbonyldiimidazole |
| | N,N′-disuccinimidyl |
| | carbonate and N- |
| | hydroxysuccinimidyl |
| | chloroformate |
| | Oxidation with periodate |
| | Enzymatic oxidation |
| | Alkyl halogens |
| | Isocyanates |
| Aldehydes & Ketones | Hydrazine derivatives |
| | Schiff base formation |
| | Reductive amination |
| | Mannich condensation |

Other examples of functional groups useful for immobilization include but are not limited to metal oxide groups; amide groups; epoxide groups; isocyanate groups; phosphate groups; sulphate groups; 1,2-dihydroxy groups; 2-aminoalcohol groups; 1,2-dicarbonyl groups; and guanidino groups.

Other chemical functional groups, complementary functional groups and reactions useful for purposes of the present invention are within the knowledge of the skilled artisan.

A chemical functional group useful for immobilization may be indigenous to a first member of a specific binding pair or the stent or coating. Alternatively, if a desirable functional group is not indigenous a novel group may be introduced to a first member or the stent or coating material using chemical techniques known to those of skill in the art.

Sulfhydryl groups can be introduced by thiolation reactions that modify amines, aldehydes, ketones, carboxylates, phosphates, and other groups. Sulfhydryl groups can also be generated by reduction of indigenous disulfides.

Carboxylate groups can be introduced by reactions that modify amines, sulfhydryls, hydroxyls, and other groups.

Primary amine groups can be introduced by reactions that modify carboxylates, sulfhydryls, carbohydrates, alkylphosphates, aldehydes, and other groups.

Arylamines can be introduced by reactions modifying phenolic compounds, such as a tyrosine side chain.

Aldehyde groups can be introduced by reactions that modify sugars, glycols and carbohydrates, amines, and other groups.

Hydrazide groups can be introduced by reactions that modify aldehydes, carboxlylates, alkylphosphates, and other groups.

According to the knowledge of the skilled artisan, it may be necessary to apply protective groups to chemically sensitive moieties to preserve their function before introducing novel chemical functional groups. This may be particularly important in the context of introducing novel functional groups into biomolecules serving as first members of a specific binding pair.

As an example of the creation of a new functional group for purposes of immobilizing a first member of a specific binding pair, sugar moieties indigenous to a biomolecule, such as heparin, possessing a 1,2-dihydroxy functional group are converted to an aldehyde group by oxidation, e.g., with sodium periodate. When the oxidized sugar contacts a primary amine group present either on the stent, or a coating material, an imine is formed between the aldehyde and amine by condensation. Reduction, e.g., using sodium cyanoborohydride converts the unstable imine to a stable secondary amine, thereby completing the formation of a stable covalent bond between the first member of a specific binding pair (i.e., heparin) and the stent or coating. Aldehydes can also be formed by oxidation of 2-aminoalcohol moieties, such as those possessed by a N-terminal serine residue, a N-terminal threonine residue, or a 5-hydroxylysine residue.

Additional information concerning chemical functional groups and reactions useful for creating bonds for immobilizing a first member of a specific binding pair to a stent or coating material is found in “Bioconjugate Techniques,” G. T. Hermanson, Academic Press, Inc., San Diego, 1996, which is incorporated herein by reference in its entirety.

For immobilization of a first member of a specific binding pair to a stent, the immobilizing bond may be formed directly with the material of which the stent is fabricated. As an example of direct binding, protein molecules are known to be capable of tightly associating with metallic gold. While not wishing to be bound by theory, it is believed that protein association is due to ionic interactions between the negatively charged gold and positively charged sites on the protein molecule, hydrophobic interactions, and formation of dative type covalent bonds between gold and free sulfhydryl groups, if present. Other types of direct binding are within the knowledge of the skilled artisan.

Alternatively, the bond may be formed with functional groups that are themselves bonded to the stent material. Such functional groups may be indigenous to the stent material (e.g., metal oxides), or they may be added de novo by the skilled artisan. Adding new functional groups, or increasing the concentration of indigenous groups may be accomplished using techniques within the knowledge of the skilled artisan, including techniques used to activate the stent surface, discussed above.

According to another alternative, the stent surface may be derivatized by bonding to the stent chemical moieties that possess chemical functional groups capable of forming a bond with a first member of a specific binding pair.

An exemplary method for derivatization is to treat the stent with organosilane compounds possessing reactive aminoalkyl groups. Upon hydrolysis of the aminosilanes in water, the resulting silanols react with the metal surface to form stable bonds. The free amino groups are then available for reaction with appropriate functional groups of the first member of the specific binding pair. Examples of aminosilanes that can be used to derivatize the stent include, but are not limited to entries in the following Table 5.

TABLE 5

3-aminopropyltriethoxysilane
3-aminopropyltrimethoxysilane
N-beta-(aminoethyl)-γ-aminopropyltrimethoxysilane
2-aminoundecyltrimethoxysilane
aminophenyltrimethoxysilane
N-(2-aminoethyl-3-aminopropyl)trimethoxysilane
trimethoxysilylpropyldiethylenetriamine
Dow Corning MDX4-4159

Other techniques for derivatizing the stent with chemical reactive moieties are within the knowledge of the skilled artisan.

If, according to the knowledge and requirements of the skilled artisan, the stent has been coated, e.g., with a polymer, the coating material may possess indigenous functional groups useful for immobilizing a first member of a specific binding pair. However, if deemed desirable, additional and/or different functional groups can be added to the coating material according to the knowledge of the skilled artisan.

For example, just as the stent material can be derivatized with amine bearing chemical moieties, the coating material can be aminated, methods for which are known to the skilled artisan. Amination results in the formation of primary and higher order amine groups on a polymer or other material. To effect amination, the polymer coated stent may be treated with gaseous plasma containing nitrogen atoms. Such a plasma may comprise ammonia gas, or organic amines, or mixtures of ammonia and organic amines excited by microwave radiation, or other energy sources known to the skilled artisan. Examples of organic amines that can be used for amination include, but are not limited to entries in the following Table 6.

TABLE 6

methylamine
dimethylamine
ethylamine
diethylamine
ethylmethylamine
n-propylamine
allylamine
isopropylamine
n-butylamine
n-butylmethylamine
n-amylamine
n-hexylamine
2-ethylhexylamine
ethylenediamine
1,4-butanediamine
1,6-hexanediamine
cyclohexylamine
n-methylcyclohexylamine
ethyleneimine

Other techniques for adding additional and/or new chemical functional groups to coating materials, including polymers are within the knowledge of the skilled artisan.

As a further example of the derivatization of a stent or coating material for purposes of providing functional groups useful for immobilizing a first member of a specific binding pair, a stent or coating bearing free hydroxyl groups is treated with a solution of tresyl chloride and pyridine to effect conversion of the hydroxyl groups to tresylate groups. Subsequently, the stent or coating is contacted with a solution containing a first member of a specific binding pair possessing a free amine group, under conditions promoting exchange of the tresylate group for the amine. In this manner, the first member of the specific binding pair is covalently attached by N-methylation to the stent or coating. If the first member possesses a single amine group susceptible to the reaction, it will become bound in a single orientation. The existence of additional amine groups increases the degrees of freedom with which the first member can become covalently bound to the stent or coating.

According to an alternative embodiment, linker 12 or first member 10 of the specific binding pair is immobilized to a thin expandable film that lines the inner surface and/or outer surface of the stent. Techniques for immobilizing first member 10 to the film are within the knowledge of the skilled artisan.

The sleeve comprises a polymeric or other material that has the property of expanding with the stent as the stent expands during implantation. Examples of polymers useful for purposes of the present invention include, but are not limited to polytetrafluoroethylene (PTFE or Teflon®), nylons and polyethylene terephthalate (Dacron®). Techniques for affixing the expandable film to the stent are within the knowledge of the skilled artisan. Examples include but are not limited to sandwiching the film between two stents and thermally spot welding the film to the underlying stent material.

Stent 100 is implanted in an artery of a human or animal patient according to techniques well known in the art. According to the knowledge of the skilled artisan, stents may be implanted after a plaque has been treated by balloon angioplasty, by atherectomy, or by other techniques for reducing plaque volume. Alternatively, a stent may be implanted simultaneous with angioplasty whereby the plaque is compressed against the wall of the artery by the expanding stent.

When stent 100 is balloon-expandable, the skilled artisan mounts the stent to the balloon of a balloon catheter. Alternatively, the stent may be premounted by a manufacturer. The balloon catheter-stent combination is then placed into arterial system of a patient in need of stent-mediated therapy, whereafter it is advanced, employing angiographic or other techniques, through the arterial system to the location of the lesion.

When the stent is properly oriented with respect to the plaque lesion, the skilled artisan causes the balloon to be expanded, which in turn causes the stent to expand. If the plaque was treated prior to stent implantation the stent is expanded sufficiently to ensure its contact with and retention by the walls of the artery, after which the balloon is deflated and the catheter removed from the patient. In this manner, the stent of the instant invention is left indwelling, providing structural support and other beneficial effects to the diseased artery, as discussed below.

Alternatively, if the lesion was not treated prior to implantation, the stent is expanded to compress the plaque sufficiently to ensure that adequate bloodflow though the artery is obtained. Thereafter, as before, the balloon is deflated and the catheter removed from the patient.

As an alternative to implanting a balloon-expandable stent, a self-expanding stent may be also be implanted according to the knowledge of the skilled artisan. Usually, self-expanding stents are implanted after a lesion has been treated by angioplasty, atherectomy, or some other treatment technique.

Other techniques for stent implantation may be employed according to the knowledge of the skilled artisan.

Arteries in which the stent and methods of the present invention may be beneficially employed for treatment include but are not limited to coronary, radial, femoral, popliteal, femoropopliteal, femoroiliac, mesenteric, carotid, aorta, renal, hepatic, tibial, iliac, pulmonary arteries, vertebro-basilar, and other arteries present in the central nervous system.

The stent and methods of the present invention may be beneficially employed for treatment of narrow lumen body cavities additional to arteries, including veins, bile ducts, ureters, urethrae, sinus ducts, eustachian tube, tear ducts, lymph ducts, bronchi, ducts or tubes that carry glandular secretions (e.g., salivary, pancreatic), ducts or tubes that carry cerebrospinal fluid within the central nervous system, spermatic ducts, fallopian tubes, and other types of ducts and tubes that exist in the human or animal patient.

Referring once again to FIG. 1, a first member 10 of a specific binding pair is any molecule or moiety immobilized to the stent to which a second member 20 of the specific binding pair is capable of specifically recognizing and binding.

“Specific binding” refers to the ability of two molecular species concurrently present in a heterogeneous (inhomogeneous) sample to bind to one another in preference to binding to other molecular species in the sample. Typically, a specific binding interaction will discriminate over adventitious binding interactions in the reaction by at least about 2-fold, 10-fold, 100-fold, 1,000-fold, 10,000-fold, 100,000-fold, or more. The affinity or avidity of binding between the first and second members of a specific binding pair is at least about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, 10⁻¹³ M, 10¹⁴ M, 10¹⁵ M, or 10¹⁶ M. Higher binding affinities as between the binding pair members are possible.

According to different embodiments of the instant invention, specific binding pairs can be chosen such that binding between the first and second members is irreversible, effectively irreversible, or reversible. Irreversible or effectively irreversible binding may be desirable if the stent is to be charged with a radioactive moiety a single time. Alternatively, reversible binding may be desirable if the stent is to be charged multiple times with a radioactive moiety.

In accordance with an embodiment of the present invention, a specific binding pair can be chosen such that the binding reaction between the first and second members results in the formation of one or more covalent bonds such that binding is irreversible without first cleaving those bonds. One non-limiting example would be the combination of an enzyme with a so-called “suicide substrate,” wherein the enzyme effects the formation of at least one covalent bond between the substrate and one or more amino acids in the active site or elsewhere in the enzyme.

Alternatively, for other specific binding pairs, the binding reaction can be of such high affinity, despite the formation of no covalent bonds, that binding is effectively irreversible (e.g., binding between avidin and biotin).

However, binding pairs can be chosen with lower, yet still high binding affinity such that the skilled artisan can effect the separation of the pair, e.g., by exposure to a soluble competitor molecule. According to the knowledge of the skilled artisan, other specific binding pairs can be treated to change the conformation of one or both members so as to effect their separation, e.g., by changing pH. For yet other specific binding pairs, even if binding is irreversible, removal or all or a portion of the complex from the stent is possible by treatment with enzymes or chemicals. For example, specific enzyme recognition sites can be introduced into proteins, nucleic acids, carbohydrates and lipids.

The first and second members of the specific binding pair are not limited in their nature or composition except that the first member be biocompatible when immobilized to the stent and/or when bound by the second member, and that the second member be biocompatible when bound to the first member. Biocompatibility may be determined empirically and indicates that the molecule in question is substantially nontoxic and nonimmunogenic. However, in circumstances wherein the second member of the specific binding pair is expected to remain bound to the first member transiently, a greater degree of immunogenicity possessed by the second member may be acceptable, particularly if it is not expected that the second member, or a closely related molecule, will be reintroduced into the patient.

As used herein, non-immunogenic means tending not to induce synthesis of specific antibodies by the patient's B cells, nor induce a cellular immune response by stimulating the patient's T cells.

The first and second members of the specific binding pair are also usefully highly specific for each other. As a corollary, the second member usefully will tend not to bind tightly to any moiety accessible to it within the vascular system, or within the human patient generally. In this way, the propensity for the second member to bind to structures not associated with the stent is minimized.

As appreciated by the skilled artisan, in many circumstances, each of molecules (or entities) comprising a specific binding pair can serve alternatively as the first or second member. Thus, as discussed herein, any class or species of molecular entity noted as being suitable to serve as the first member immobilized to the stent oftentimes can serve as the second member, in which case the complementary molecule of the binding pair then serves as the first member. Determination of which molecular entity of a specific binding pair advantageously serves as the first member is within the knowledge of the skilled artisan.

Frequently, but not necessarily, first member 10 is a natural or non-natural biomolecule of human or non-human origin. Thus, first member 10 can be a natural product derived from a cell or an organism; a genetically engineered molecule not ordinarily found in nature, but that is capable of being produced by a cell or organism, or by an in vitro synthetic process; an enzymatically or chemically modified natural product or engineered molecule; or be comprised of fragments of natural and/or genetically engineered molecules joined enzymatically or chemically, either in man-made reaction vessels, or in genetically modified cells or organisms. Alternatively, first member 10 can be completely synthetic and non-natural. According to yet another alternative, first member 10 can be a supramolecular structure, including a virus (e.g., bacteriophage), or other assemblage of different kinds of biomolecules.

Examples of classes of biomolecules of which the first member of the specific binding pair can be comprised include, but are not limited to protein, nucleic acid, carbohydrate, lipid, and accessory or prosthetic groups (e.g., heme, chlorophyll, vitamins).

Carbohydrate includes oligosaccharides and polysaccharides, branched or unbranched, comprising a single species or different species of monosaccharide precursors. Carbohydrate can be pure carbohydrate, or can be found conjugated to protein (glycoprotein and proteoglycan), or conjugated to lipids (glycolipid).

Lipid includes triacylglycerols, waxes, phosphoglycerides, sphingolipids, and sterols (e.g., cholesterol and derivatives of cholesterol, including steroid hormones).

As appreciated by the skilled artisan, many biomolecules are susceptible to degradation by cells and/or enzymes that exist within a patient's circulatory system. Thus, in some circumstances it is advantageous to modify the biomolecule in some fashion to protect it from degradation. Protective modifications suitable for this purpose are within the knowledge of the skilled artisan. For example, proteolytic cleavage sites in proteins may be destroyed by mutation, and the phosphate backbone of a nucleic acid may be replaced by phosphothioate moieties so as to increase resistance to nucleases.

As used herein, the terms “protein”, “polypeptide”, and “peptide” are used interchangeably to refer to a naturally-occurring or synthetic polymer of amino acid monomers (residues), irrespective of length, where amino acid monomer here includes naturally-occurring amino acids, naturally-occurring amino acid structural variants, and synthetic non-naturally occurring analogs that are capable of participating in peptide bonds. The terms “protein”, “polypeptide”, and “peptide” explicitly permits of post-translational and post-synthetic modifications, such as glycosylation, phosphorylation, and farnesylation.

The term “oligopeptide” herein denotes a protein, polypeptide, or peptide having 25 or fewer monomeric subunits.

As used herein, “nucleic acid” includes polynucleotides having natural nucleotides in native 5′-3′ phosphodiester linkage—e.g., dexyribonucleic acid (DNA) or ribonucleic acid (RNA)—as well as polynucleotides that have normatural nucleotide analogues, normative internucleoside bonds, or both, so long as the normatural polynucleotide is capable of sequence-discriminating basepairing under experimentally desired conditions.

Unless otherwise specified, the term “nucleic acid” includes any topological conformation; the term thus explicitly comprehends single-stranded, double-stranded, partially duplexed, triplexed, hairpinned, circular, and padlocked conformations.

As used herein, “nucleic acid” encompasses “oligonucleotides,” nucleotide polymers comprising between 2 and about 200 nucleotides, more typically about 20-100 nucleotides.

However, according to the needs of the skilled artisan, nucleic acid polymers of much greater length can be advantageously used for purposes of the present invention. Nucleic acids with as few as about 10,000, 5,000, 2,500, 1,000, 500, or 200 nucleotides may be used. Alternatively, nucleic acids with as many as about 25,000, 50,000, 100,000, 250,000, 500,000, or 1,000,000 nucleotides may be used.

Examples of specific binding pairs include, but are not limited to the entries in the following Table 7. As mentioned above, according to the knowledge of the skilled artisan any entry listed as a first member can oftentimes serve as the second member, and vice versa.

TABLE 7
Specific Binding Pairs

| First Binding Pair Member | Second Binding Pair Member |
|---|---|
| Lectin | Carbohydrate |
| Receptor | Ligand |
| Receptor | Hormones, including |
| | proteinaceous |
| | (e.g., insulin), small |
| | molecule (e.g., |
| | epinephrine) and lipid |
| | soluble (e.g., estrogen) |
| | hormones |
| Receptor | Neurotransmitters |
| | including small molecule |
| | (e.g., acetylcholine) and |
| | proteinaceous (e.g., |
| | neuropeptide Y) |
| | neurotransmitters |
| Enzyme | Non-cleavable substrates |
| Antibody | Antigen, Epitope, Hapten, |
| | Protein, Nucleic Acid, |
| | Lipid, Carbohydrate |
| Protein A | IgG and other antibodies |
| Protein G | IgG and other antibodies |
| DNA | RNA with complementary |
| | sequence |
| DNA | DNA with complementary |
| | sequence |
| DNA | Sequence-specific DNA |
| | binding protein (e.g., |
| | transcription factor DNA |
| | binding domain; zinc |
| | fingers, leucine zipper, |
| | homeobox domain, winged |
| | helix domain, etc.) |
| DNA | Nonsequence-specific DNA |
| | binding protein (e.g., |
| | histone) |
| RNA | Sequence-specific RNA |
| | binding protein (e.g., |
| | transcription factor) |
| RNA | Nonsequence-specific RNA |
| | binding protein that |
| | recognizes a RNA secondary |
| | or tertiary structure |
| | (e.g., as found in |
| | transfer or ribosomal RNA) |
| RNA | RNA that binds RNA by |
| | virtue of recognizing a |
| | RNA secondary or tertiary |
| | structure (e.g., as found |
| | in transfer or ribosomal |
| | RNA) |
| Cell adhesion molecule | Cell adhesion molecule |
| (e.g., cadherins, | substrate |
| integrins, neural cell | |
| adhesion molecules (NCAM)) | |
| Protein interaction domain | Complementary protein |
| | interaction domain |
| Avidin, streptavidin, and | Biotin |
| related proteins | |
| Heparin | Protamine |
| Protein | Protein, Nucleic Acid, |
| | Lipid, Carbohydrate |
| Nucleic Acid | Protein, Nucleic Acid, |
| | Lipid, Carbohydrate |
| Lipid | Protein, Nucleic Acid, |
| | Lipid, Carbohydrate |
| Carbohydrate | Protein, Nucleic Acid, |
| | Lipid, Carbohydrate |

Techniques for the production of the molecular entities that will serve as the first or second member of the specific binding pair are well known in the art, and include chemical synthetic methods. An alternative method is to purify such molecular entities from genetically engineered cells.

According to a useful embodiment of the present invention, the first member of the specific binding pair is a protein or glycoprotein, polypeptide, oligopeptide, or peptide, and the second member is an antibody, or related antigen or epitope binding protein that specifically recognizes and binds the protein.

As used herein, the term “antibody” refers to a polypeptide, at least a portion of which is encoded by at least one immunoglobulin gene, or fragment thereof, and that can bind specifically to a desired target molecule. The term includes naturally-occurring forms, as well as fragments and derivatives. Naturally occurring antibodies include IgA, IgD, IgE, IgG, and IgM. Subclasses include IgA1, IgA2, IgG1, IgG2, IgG3 and IgG4.

Fragments within the scope of the term include those produced by digestion with various proteases, those produced by chemical cleavage and/or chemical dissociation, and those produced recombinantly, so long as the fragment remains capable of specific binding to a target molecule. Among such fragments are Fab, Fab′, Fv, F(ab)′₂, “half-antibodies” formed by reduction of the disulfide bonds joining an immunoglobulin's heavy chains, and single chain Fv (scFv) fragments.

Derivatives within the scope of the term include antibodies (or fragments thereof) that have been modified in sequence, but remain capable of specific binding to a target molecule, including: interspecies chimeric and humanized antibodies; antibody fusions; heteromeric antibody complexes and antibody fusions, such as diabodies (bispecific antibodies), single-chain diabodies, and intrabodies (see, e.g., Marasco (ed.), “Intracellular Antibodies: Research and Disease Applications,” Springer-Verlag New York, Inc. (1998) (ISBN: 3540641513), the disclosure of which is incorporated herein by reference in its entirety).

As used herein, antibodies can be produced by any known technique, including harvest from cell culture of native B lymphocytes, harvest from culture of hybridomas, recombinant expression systems, and phage display.

As used herein, “antigen” refers to a ligand that can be bound by an antibody; an antigen need not itself be immunogenic. The portions of the antigen that make contact with the antibody are denominated “epitopes”.

Additional information about antibodies, including their production, purification and radiolabeling, as well as information about antigens, epitopes and haptens can be found in “Current Protocols in Immunology,” J. E. Coligan, et al., Editors, John Wiley & Sons, Inc., New York, N.Y., 2001, which is incorporated herein by reference in its entirety.

As understood by the skilled artisan, numerous protein or peptide sequences can serve as the first member of the specific binding pair immobilized to the stent. Such peptides should be substantially nontoxic and nonimmunogenic when bound to the stent and/or when bound by the antibody. That is, the peptide may possess some degree of toxicity or immunogenicity when freely soluble; however, what is important for purposes of the present invention is that the toxicity or immunogenicity be reduced to negligible or acceptable levels when the peptide is either immobilized to the stent or otherwise sequestered by virtue of being bound by the antibody or other second member of the specific binding pair.

According to an especially useful embodiment of the present invention the first member of the specific binding pair is a relatively short peptide sequence, called an epitope tag, to which a known antibody binds with high specificity and affinity. Many such antibodies are commercially available. According to the knowledge of the skilled artisan, new epitope tags can be developed by raising antibodies against randomized or semi-randomized peptide sequences, and then selecting for antibodies that bind to the tag sequence, and bind negligibly to cells and tissues under non-stringent conditions.

Typically, epitope tags occur in lengths of about 5-15 amino acid residues. While not wishing to be bound by theory, it is likely that due to their short length, epitope tags do not form extensive secondary or tertiary structures and can be immobilized at high density on the stent. A further advantage of epitope tags is that they rarely occur as constituents of extracellular proteins. This reduces the degree of adventitious binding by their complementary antibodies.

Epitope tags are more fully described in “Epitope tagging: general methods for tracking recombinant proteins,” C. E. Fritze and T. R. Anderson, Methods in Enzymology (2000) 327:3-16, incorporated by reference herein in its entirety. Examples of epitope tags useful for purposes of the present invention include, but are not limited to the entries from the following Table 8.

TABLE 8

| | Tag Sequence | |
|---|---|---|
| | (One Letter | |
| Tag Name | Protein Code) | Sequence ID No. |
| HA | YPYDVPDYA | SEQ ID NO: l |
| Myc | EQKLISEEDL | SEQ ID NO: 2 |
| FLAG | DYKDDDK | SEQ ID NO: 3 |
| Polyhistidine | HHHHHH | SEQ ID NO: 4 |
| (6-His) | | |
| AU1 | DTYRYI | SEQ ID NO: 5 |
| AU5 | TDFYLK | SEQ ID NO: 6 |
| IRS | RYIRS | SEQ ID NO: 7 |
| B-Tag | QYPALT | SEQ ID NO: 8 |
| Universal | HTTPHH | SEQ ID NO: 9 |
| S-Tag | KETAAAKFERQHMDS | SEQ ID NO: 10 |
| Protein C | EDQVDPRLIDGK | SEQ ID NO: 11 |
| Glu-Glu | EYMPME or | SEQ ID NO: 12 |
| | EFMPME | SEQ ID NO: 13 |
| KT3 | PPEPET | SEQ ID NO: 14 |
| VSV | MNRLGK | SEQ ID NO: 15 |
| T7 | MASMTGGQQMG | SEQ ID NO: 16 |
| HSV | QPELAPEDPED | SEQ ID NO: 17 |

When an epitope tag serves as the first member of the specific binding pair, an antibody that specifically binds the epitope tag usefully serves as the second member of the specific binding pair. However, as appreciated by the skilled artisan, the antibody may serve as the first member immobilized to the stent, whereas the epitope tag can serve as the second member.

Another peptide sequence, not expressed in normal tissue, useful as the first member of a specific binding pair for purposes of the present invention is found at the junction of an oncogenic deletion occurring in a mutated form of the human epidermal growth factor receptor (EGFR), designated EGFRvIII. The deletion comprises 802 nucleotides and results in an in-frame deletion of protein coding sequence with the formation of a novel glycine residue at the deletion junction. The peptide comprises the amino acid sequence Leu-Glu-Glu-Lys-Lys-Gly-Asn-Tyr-Val-Val-Thr-Asp-His-Cys (SEQ ID NO:18), wherein each amino acid is represented by its three-letter code.

Polyclonal, monoclonal, and single-chain antibodies that recognize and specifically bind the EGFRvIII peptide have been generated, as discussed in C. J. Wikstrand, et al., Cancer Res., Vol. 55, pp. 3140-3148, Jul. 15, 1995; I. A. Lorimer, et al., Clin. Cancer Res., Vol 1, pp. 859-864, August 1995; P. A. Humphrey, et al., Proc. Natl. Acad. Sci. USA, Vol. 87, pp. 4207-4211, June 1990; I. A. Lorimer, et al., Proc. Natl. Acad. Sci. USA, Vol. 93, pp. 14815-14820, December 1996, each of which is incorporated herein by reference in its entirety. Such antibodies and antibody fragments are usefully employed as the second member of the specific binding pair for purposes of the present invention. According to an alternative embodiment, the anti-EGFRvIII antibody may serve as the first member of the specific binding pair immobilized to the stent, whereas the peptide serves as the second member of the specific binding pair.

Techniques for the production of epitope tags and other peptides and proteins that will serve as the first or second member of the specific binding pair are well known in the art, and include chemical synthetic methods. An alternative method is to purify such peptides and proteins from genetically engineered cells.

Referring once again to FIG. 1, the second member 20 of the specific binding pair binds the first member 10. As discussed above, second member 20 is a molecular entity capable of specifically recognizing and binding the first member. Also as discussed above, according to a useful embodiment of the present invention second member 20 is an antibody or related molecule, whereas first member 10 is a peptide or protein. According to the knowledge of the skilled artisan, many other types of molecular entities can comprise the specific binding pair, examples of which are listed in Table 7, herein above, entitled “Specific Binding Pairs.”

Second member 20 is directly associated with or connected to radioactive moiety 30, or is connected via a molecular linker 22 to the radioactive moiety 30. In this manner, second member 20 is rendered radioactive for purposes of inhibiting restenosis, or neointimal hyperplasia by the release of radioactive decay products 40. Linker 22 may be the same or different from linker 12. Linker molecules appropriate for connecting second member 20 to radioactive moiety 30 are well known in the art.

According to one embodiment, radioactive moiety 30 consists of a radioactive atom and may be joined to second member 20 by a covalent or non-covalent bond. The radioactive atom may replace a nonradioactive atom indigenous to the native structure of the second member, or may be present adventitiously, i.e., at a locus where a corresponding nonradioactive atom would not typically be present in the native structure.

As used herein, and unless otherwise indicated, the term “atom” is inclusive of uncharged atoms, as well as atoms, as well as atoms having a positive or negative charge of unity or greater, i.e., ions.

According to an alternative embodiment, radioactive moiety 30 comprises a molecular entity wherein a radioactive atom replaces a nonradioactive atom indigenous to the native structure of the entity. The radioactive moiety may also comprise a molecular entity wherein a radioactive atom is present adventitiously.

According to yet another embodiment, radioactive moiety 30 comprises a chelating group that binds a radioactive atom.

Useful for purposes of the present invention are radioactive atoms that decay with the emission of beta particles, alpha particles, positrons, x-rays, gamma rays, or Auger electrons, and other decay products. Especially useful for purposes of the present invention are those isotopes that decay with half-lives of about 3 hours to about 300 days, although radioisotopes with shorter or longer half-lives may also be used according to the knowledge and requirements of the skilled artisan. Such isotopes decay to negligible levels in about 1 day to 4 years. Radioisotopes for purposes of the present invention usefully are obtained in solution in water or other polar fluid in elemental form (i.e., uncharged) or ionic form. As appreciated by the skilled artisan, when in ionic form, radioisotopes may occur in various different valence states, as anions, or as cations, depending upon the particular radioisotope being considered.

Examples of radioisotopes useful for purposes of the present invention include, but are not limited to the following: 7Be, 24Na, 28Mg, 32P, 33P, 35S, 37Ar, 42K, 43K, 45Ca, 47Ca, 43Sc, 44Sc, 44mSc, 46Sc, 47Sc, 48Sc, 45Ti, 48V, 48Cr, 51Cr, 52Mn, 52Fe, 59Fe, 55Co, 56Co, 57Co, 58Co, 58mCo, 56Ni, 57Ni, 66Ni, 61Cu, 64Cu, 67Cu, 62Zn, 65Zn, 69mZn, 71mZn, 72Zn, 66Ga, 67Ga, 72Ga, 73Ga, 68Ge, 69Ge, 71Ge, 77Ge, 71As, 72As, 73As, 74As, 76As, 77As, 72Se, 73Se, 75Se, 76Br, 77Br, 80mBr, 82Br, 76Kr, 79Kr, 85mKr, 81Rb, 82mRb, 83Rb, 84Rb, 86Rb, 82Sr, 83Sr, 85Sr, 89Sr, 91Sr, 85mY, 86Y, 87Y, 87mY, 88Y, 90Y, 90mY, 91Y, 92Y, 93Y, 86Zr, 88Zr, 89Zr, 95Zr, 97Zr, 90Nb, 91mNb, 92mNb, 95Nb, 95mNb, 96Nb, 90Mo, 93mMo, 99Mo, 94Tc, 95Tc, 95mTc, 96Tc, 97mTc, 99mTc, 97Ru, 103Ru, 105Ru, 99Rh, 99mRh, 100Rh, 101mRh, 102Rh, 105Rh, 100Pd, 101Pd, 103Pd, 109Pd, 111mPd, 112Pd, 105Ag, 106mAg, 110mAg, 111Ag, 112Ag, 113Ag, 107Cd, 115Cd, 115mCd, 117mCd, 109In, 110In, 111In, 114mIn, 115mIn, 110Sn, 113Sn, 117mSn, 119mSn, 121Sn, 123Sn, 125Sn, 118mSb, 119Sb, 120mSb, 122Sb, 124Sb, 126Sb, 127Sb, 128Sb, 129Sb, 118Te, 119Te, 119mTe, 121Te, 121mTe, 123mTe, 125mTe, 127Te, 127mTe, 129mTe, 131mTe, 132Te, 123I, 124I, 125I, 126I, 130I, 131I, 133I, 135I, 122Xe, 125Xe, 127Xe, 129mXe, 131mXe, 133Xe, 133mXe, 135Xe, 127Cs, 129Cs, 131Cs, 132Cs, 136Cs, 128Ba, 131Ba, 133mBa, 135mBa, 140Ba, 132La, 133La, 135La, 140La, 141La, 132Ce, 133mCe, 134Ce, 135Ce, 137Ce, 137mCe, 139Ce, 141Ce, 143Ce, 144Ce, 139Pr, 142Pr, 143Pr, 145Pr, 138Nd, 139mNd, 140Nd, 147Nd, 143Pm, 148Pm, 148mPm, 149Pm, 151Pm, 153Sm, 156Sm, 145Eu, 146Eu, 147Eu, 148Eu, 149Eu, 150mEu, 152mEu, 156Eu, 157Eu, 146Gd, 147Gd, 149Gd, 151Gd, 153Gd, 159Gd, 149Tb, 150Tb, 151Tb, 152Tb, 153Tb, 154Tb, 154mTb, 154m2Tb, 155Tb, 156Tb, 156mTb, 156m2Tb, 160Tb, 161Tb, 153Dy, 155Dy, 157Dy, 159Dy, 166Dy, 160mHo, 166Ho, 167Ho, 160Er, 161Er, 165Er, 169Er, 171Er, 172Er, 165Tm, 166Tm, 167Tm, 168Tm, 170Tm, 172Tm, 173Tm, 166Yb, 169Yb, 175Yb, 169Lu, 170Lu, 171Lu, 172Lu, 174mLu, 176mLu, 177Lu, 177mLu, 179Lu, 170Hf, 171Hf, 173Hf, 175Hf, 179m2Hf, 180mHf, 181Hf, 184Hf, 173Ta, 175Ta, 176Ta, 177Ta, 180Ta, 182Ta, 183Ta, 184Ta, 178W, 181W, 185W, 187W, 188W, 181Re, 182Re, 182mRe, 183Re, 184Re, 184mRe, 186Re, 188Re, 189Re, 190mRe, 182Os, 183Os, 183mOs, 185Os, 189mOs, 191Os, 191mOs, 193Os, 184Ir, 185Ir, 186Ir, 187Ir, 188Ir, 189Ir, 190Ir, 190m2Ir, 192Ir, 193mIr, 194Ir, 194m2Ir, 195mIr, 188Pt, 189Pt, 191Pt, 193mPt, 195mPt, 197Pt, 200Pt, 202Pt, 191Au, 192Au, 193Au, 194Au, 195Au, 196Au, 196m2Au, 198Au, 198mAu, 199Au, 200mAu, 192Hg, 193Hg, 193mHg, 195Hg, 195mHg, 197Hg, 197mHg, 203Hg, 198Tl, 199Tl, 200Tl, 201Tl, 202Tl, 200Pb, 201Pb, 202mPb, 203Pb, 209Pb, 212Pb, 203Bi, 204Bi, 205Bi, 206Bi, 210Bi, 204Po, 206Po, 207Po, 210Po, 209At, 210At, 211At, 211Rn, 222Rn, 223Ra, 224Ra, 225Ra, 225Ac, 226Ac, 228Ac, 227Th, 231Th, 234Th, 228 Pa, 229 Pa, 230 Pa, 232 Pa, 233 Pa, 234 Pa, 230U, 231U, 237U, 240U, 234Np, 236mNp, 238Np, 239Np, 234Pu, 237Pu, 243Pu, 245Pu, 246Pu, 247Pu, 239Am, 240 Am, 242Am, 244Am, 240 Cm, 241 Cm, 242 Cm, 252 Cm, 243Bk, 244Bk, 245Bk, 246Bk, 248mBk, 250Bk, 246Cf, 247Cf, 253Cf, 254Cf, 250Es, 251Es, 253Es, 254Es, 254mEs, 255Es, 256mEs, 251Fm, 252Fm, 253Fm, 254Fm, 255Fm, 257Fm, 257Md, 258Md, 260Md and 262Lr.

Additional radioactive isotopes useful for purposes of the present invention include, but are not limited to: 11C, 13N, 14C, 15O, 18F, 81mKr, 137Cs, 212Bi, 62Cu, 120Sb, 68Ga, 103mRh, 82Rb, 204Tl, 132I, 165Dy, and 60Co. Other radioisotopes useful for purposes of the present invention are within the knowledge of the skilled artisan.

Criteria for choosing a radioisotope to be used in the present invention are well known to the skilled artisan, and include, but are not limited to the type and energy of radioactive decay product yielded by the isotope; the half-life of decay; chemical properties of the atom or ion; and the biological and toxicological properties of the atom or ion.

The decay product(s) yielded by the decay of the radioisotope should be of sufficiently high energy, and sufficiently low mass such that the decay products, whether particles or photons, can reach and penetrate the nuclei of the cells of the intima in the vicinity of the stent. The characteristics of the decay products should be such that they are capable of interacting with cells in such a way as to inhibit or interfere with biological processes necessary for cellular replication, or that cause the cell to undergo apoptosis. Usually, the energy is not so high that the decay products reach tissues far away from the stent, or reach persons in close proximity to the patient. However, there may be circumstances wherein high energy, highly penetrative decay products are desirable, according to the knowledge and requirements of the skilled artisan.

Half-life is chosen with consideration to the length of time desirable for irradiation of intimal cells. Usually, a half-life of decay is not chosen that is so short that radiation falls to ineffective levels before intimal cells are growth inhibited. Likewise, a half-life of decay is not chosen that is so long that radiation persists at a high level after such time that intimal cells are substantially or completely growth inhibited. In this manner, deleterious side effects of irradiation are minimized. However, according to certain embodiments of the present invention, the radioactive moiety joined to the second member of the specific binding pair can be removed from the stent, e.g., after a fully therapeutic dose of radiation has been absorbed by the intimal cells. In such circumstances, the radioisotope chosen can have a longer half-life than would be considered optimal if the radioactive moiety were not to be removed.

The radioisotope chosen should be chemically compatible with the instant invention. For example, the radioisotope, in its elemental form, or in its ionic form should not be so reactive that it changes the structure or function of any component of the instant invention in a way that impairs its usefulness. Rather, the chemical properties should be such that the radioisotope can be stably incorporated into the molecular entity joined to the second member 20 of the specific binding pair (thus forming together the radioactive moiety 30), or alternatively, chelated by a chelating group that serves a similar function as the molecular entity.

Amenability of the radioisotope to participate in chemical reactions that effect its incorporation into a molecular entity, or the affinity with which it is bound by a particular chelating group are also factors that may be considered.

The radioisotope chosen should also be biologically and toxicologically compatible with the patient in whom the stent is implanted and subsequently rendered radioactive. Thus, less useful for purposes of the present invention are radioisotopes that are highly toxic or otherwise harmful to the patient if it should be released from the stent.

Other criteria for choosing a radioisotope for purposes of the present invention are within the knowledge of the skilled artisan.

As will be appreciated by the skilled artisan, more than one radioisotope may be chosen and used for purposes of the present invention. Thus, of the radioactive moieties indirectly immobilized on a stent, all may comprise a single species of radioisotope, or the population may be comprised of a plurality of species of radioisotope combined in proportions according to the knowledge and need of the skilled artisan. In this manner the useful properties of different radioisotopes can be combined. For example, a single radioisotope decays at a linear rate. By combining radioisotopes of different half-lives, it is possible to make a stent radioactive such that the radioactivity decays non-linearly, if so desired by the skilled artisan.

By combining different radioisotopes it is also possible to control and define the types of radioactive decay products absorbed by the tissues. For example, a beta particle emitting radioisotope can be combined with a gamma emitting radioisotope. Other useful combinations will be apparent to the skilled artisan.

According to the knowledge of the skilled artisan, the total dose of radiation desirable to be absorbed by the cells of artery in the vicinity of the stent is determined by the severity of the disease to be treated, as well as other factors.

Controlling the radiation dose can be accomplished in different ways, as will be appreciated by the skilled artisan.

A useful method for controlling dose is to choose a radioisotope that decays to negligible levels at about the same time that a predetermined dose has been absorbed by the artery. Dose will therefore be a function of the total number of radioactive atoms bound to the stent, whereas half-life determines the time period in which the dose is absorbed (intensity).

The number of radioactive atoms bound to the stent can be controlled in various ways according to the knowledge and requirements of the skilled artisans. Variables include, but are not limited to the number of radioactive atoms per radioactive moiety; the number of radioactive moieties per second member of the specific binding pair; the number of second members of the specific binding pair bound to the stent; and the number of first members of the specific binding pair available to be bound by the second members.

The intensity of the dose can be controlled by choosing a radioisotope that decays with a shorter or longer half-life. Thus, if it is desirable that the total dose be absorbed in a short time (greater intensity), a relatively short half-life radioisotope is chosen. In contrast, if it is desirable that the total dose be absorbed in a longer time (lower intensity), a relatively long half-life isotope is chosen. As will be apparent to the skilled artisan, if it is desirable that the dose intensity be variable over time, radioisotopes of different half-lives may be combined and indirectly immobilized to the stent. As time progresses, the intensity declines as a function of the proportion of the population of radioactive atoms represented by each of the different types of radioisotopes possessing a distinct half-life.

Dose and intensity can also be controlled by either removing radioactive moieties from a stent that has been made radioactive according to the methods of the present invention, or by renewing the radioactivity of a stent. Removing radioactivity may be desirable if a stent has been made more radioactive than necessary to effectively inhibit intimal hyperplasia. In contrast, renewing radioactivity may be desirable if a stent is insufficiently radioactive to control the disease process.

As understood by the skilled artisan, radioactivity can be removed from the stent according to different methods. For example, the second member of the specific binding pair can be separated from the first by contacting the first and/or second member with a competitor molecule that specifically competes for binding between the first and second members.

Alternatively, any component responsible for immobilizing the radioactive atom to the stent (radioactive moiety, second member, first member) can be enzymatically or chemically cleaved. Such cleavage can occur as the result of a deliberate intervention whereby it is desirable to rapidly cleave and thereby remove radioactive atoms. In such circumstances, the cleavage site has often been engineered into one or more of the immobilizing components, and cleaving enzymes or chemical agents are introduced into the patient.

However, according to an alternative embodiment, endogenous biological processes originating from the patient, usually enzymatic in nature, are relied upon to gradually degrade one or more of the immobilizing components, thereby releasing formerly bound radioactive atoms at a predictable rate.

Other methods for removing radioactivity from the stent will also be apparent to the skilled artisan.

The spatial pattern and intensity of radiation dose can be controlled as well. According to the knowledge of the skilled artisan, the pattern and/or density of immobilization of the first member of the specific binding pair on the stent can be varied. In this manner, the pattern and/or density of immobilization of the second member and radioactive moiety can be varied as well. As a result, the pattern and/or strength of radiation emitted by the stent is variable.

For example, the first member of the specific binding pair can be concentrated at the ends of the stent, the density falling off in a continuous or discontinuous pattern toward the center of the stent. Alternatively, the concentration can be highest in the center, falling off in a continuous or discontinuous pattern toward the ends. According to certain embodiments no first member of the specific binding pair is immobilized within a predetermined distance from one or both ends of the stent, although it does occur immobilized elsewhere. Other patterns of variable or uniform density of immobilization of the first member of the first binding pair are possible as will be appreciated by the skilled artisan.

According to another useful embodiment, more than one species of a first member of a specific binding pair can be immobilized, wherein the pattern and/or density with which different first members are immobilized is different as between the distinct species. In this way, through different intermediate second members, different radioisotopes with distinct half-lives and/or decay products can be immobilized to the stent in variable patterns and densities as well. Thus, for example, a beta emitting radioisotope can be concentrated at the ends of the stent, whereas a gamma emitting radioisotope can be concentrated in the center portion of the stent.

The total dose of absorbed radiation may be as low as about 10⁻³ grays (Gy), 10⁻² Gy, 10⁻¹ Gy, 1 Gy, 5 Gy, 10 Gy, 50 Gy, 100 Gy, or about 500 Gy.

Alternatively, the total dose of absorbed radiation may be as much as about 10⁻² Gy, 10⁻¹ Gy, 1 Gy, 5 Gy, 10 Gy, 50 Gy, 100 Gy, 500 Gy, or about 1000 Gy. Other radiation doses are possible according to the knowledge of the skilled artisan.

Radiation dose can also be expressed in terms of an amount of radiation, regardless of the type of source, absorbed in some defined period of time that has a quantifiable biological effect in the context of a model reference system.

Exemplary time periods include about 1 minute, 1 hour, 10 hours, 1 day, 1 week, 1month, 1 year, or multiples thereof. Exemplary biological effects include, but are not limited to percent inhibition of cell mitosis, or other indicia of cellular growth and division, or percent induction of apoptosis or programmed cell death.

Exemplary percentages of quantifiable biological effect may be as small as about 0.1%, 1%, 2.5%, 5%, 10%, 20%, 30%, 40%, 50%, 60%, 70%, 80%, or about 90%. Alternatively, percentages of quantifiable biological effect may be as great as about 100%, 90%, 80%, 70%, 60%, 50%, 40%, 30%, 20%, 10%, 5%, 2.5%, or about 1%.

Exemplary model systems include, but are not limited to a computer model, and an in vitro system wherein the viability of cells, tissues or organs, and at least some of their respective functions, are sustained outside of the animal (including human) from which they are collected. For example, a section of artery can be cultured and the endothelial cells injured in some fashion, such as by scraping the inner surface of the artery section, or by stretching the section in one or more dimensions. Thereafter, a stent made radioactive by the methods of the present invention can be made to contact the section for a defined time, after which a quantifiable biological effect is measured according to methods known in the art.

Model systems also include animal models of the human cardiovascular system wherein the disease process of neointimal hyperplasia is simulated or replicated, and where the stent and methods of the present invention can be tested experimentally without risk of harming a human patient. For example, pigs are considered good models of the human cardiovascular system. An artery of one or more test pigs can be injured by balloon expansion. Thereafter a stent of the present invention can be implanted, after which time the methods of the present invention are practiced to make the implanted stent radioactive. After irradiation for a defined period of time, a quantifiable biological effect is measured according to methods known in the art. Other animal models include, but are not limited to mice, rats, rabbits, dogs, and sheep.

Many techniques for rendering second member 20 of a specific binding pair radioactive are known in the art.

According to a useful embodiment of the present invention, if second member 20 is a protein containing one or more tyrosine or histidine residues, the protein may be labeled with a radioisotope of iodine, e.g., ¹²³I, ¹²⁴I, ¹²⁵I, ¹²⁹I and ¹³¹I, by treating the protein with Chloramine T, iodo-gen, iodo-beads, or other oxidizing agent, according to the techniques well known in the art. According to another embodiment, Bolton-Hunter reagent may be used to radioactively iodinate proteins where free amino groups occur (lysine or free terminal amine).

According to yet another embodiment, serine, threonine and tyrosine residues can be radioactively phosphorylated with ³²P. According to the knowledge of the skilled artisan, where such amino acids occur in the context of the appropriate recognition site, a corresponding protein kinase can be used to transfer the gamma phosphate group containing ³²P from radiolabeled ATP.

If the second member 20 of the specific binding pair is a nucleic acid it can also be radiolabeled with ³²P. According to the knowledge of the skilled artisan, T4 polynucleotide kinase can be used to transfer the gamma phosphate group containing ³²P from radioactive ATP to a DNA molecule possessing a free 5′-hydroxyl terminus. Alternatively, terminal deoxynucleotidyl transferase can be used to transfer mononucleotides containing alpha ³²P from deoxyribonucleotide triphosphates to the 3′-hydroxyl terminus of DNA. According to yet another alternative, the phosphate backbone of a nucleic acid can be made radioactive by synthesizing the nucleic acid de novo using radioactively labeled dNTPs or rNTPs. Other methods for labeling nucleic acids with radioactive atoms are known in the art.

Additional information regarding radioactive labeling of nucleic acids can be found in “Current Protocols in Molecular Biology,” F. M. Ausubel, et al., Editors, John Wiley & Sons, Inc., New York, N.Y., 2001, which is incorporated herein by reference in its entirety.

According to an alternative embodiment of the present invention, second member 20 of the specific binding pair is rendered radioactive by attaching to it a radioactive metallic atom or ion. According to the knowledge of the skilled artisan, one method for doing so is to join to second member 20 a metal atom chelating moiety which in turn binds one or more radioactive metal atoms or ions. The combination of the chelating moiety and radioactive metallic atom or ion comprises radioactive moiety 30.

While not wishing to be bound by theory, if the radioactive metal atom is a cation it can accept one or more pairs of electrons from electron-pair donors in the chelating moiety to form one or more coordinate covalent bonds. The resultant coordination complex comprises the radioactive moiety 30.

Typically, the chelating moiety is attached covalently to the second member 20, but noncovalent bonds can be used as well. Useful chelating moieties are bifunctional chelating agents (BCA) that contain a chemical-reactive group for coupling to the second member 20 and a metal-chelating group for chelating metal ions or atoms.

A second member 20 of a specific binding pair (e.g., an antibody) may be joined first to the chelating moiety. Subsequently, according to the knowledge of the skilled artisan, the complex is contacted with radioactive metal atoms, as uncharged atoms or ions, such that their binding to the chelating moiety is effected. Alternatively, the radioactive metal atoms or ions may be bound to the chelating moiety first, after which the chelating moiety-metal atom complex is joined to second member 20.

According to an alternative embodiment, a plurality of chelating moieties can be joined to each second member 20.

Metal chelating moieties that are joined to proteins for the purpose of loading the chelating moiety with radioactive metal atom are discussed in the following issued U.S. Pat. Nos. 4,454,106; 4,472,509; 4,831,175; 5,246,692; 5,250,285; 5,514,363; 5,837,218; 5,891,418; 5,922,302; 6,180,082; 6,183,721; 6,203,775 each of which is incorporated herein by reference in its entirety.

The chelating moiety may also be a chemical functional group that is integral in a biomolecule. For example, as is apparent to the skilled artisan, protein sulfhydryl groups, such as those found in reduced antibody molecules, are capable of chelating certain types of metal ions.

Examples of natural and chemical-synthetic metal atom chelating moieties include but are not limited to the entries in the following Table 9.

TABLE 9
Metal Atom or Ion Chelating Moieties

ferritin
metallothionein
ferredoxin (and other iron-sulfur proteins)
Aztobacter molybdenum-iron protein
nitrogenase
ceruloplasmin
laccase
1,4,7,10,-tetraazacyclodo-decane-N,N′,N″,N′″-tetraacetic
acid (DOTA)
2-p-nitrobenzyl-DOTA (nitrobenzyl DOTA)
triethylenetetramine-hexaacetic acid
1,2-diaminocyclohexane-N,N,N′,N′-tetra-acetic acid
N,N′-Di(2-hydroxybenzyl) ethylenediamine
N-(2-hydroxyethyl)ethylenediaminetriacetic acid
nitrilotriacetic acid
ethylene-bis(oxyethylene-nitrilo) tetraacetic acid
N1-(p-isothiocyanatobenzyl)-diethylene-triamine-N1,
N2,N3,N3-tetraacetic acid (DTTA)
1-(p-isothiocyanatobenzyl)ethylene diaminetetracetic
acid
1-(p-isothiocyanatobenzyl)diethylenetriaminepentaacetic
acid
1,4,7,10,-tetraaza-cyclododecane-N,N′,N″,-triacetic
acid
1,4,7-tris(carboxymethyl)-10-(2′-hydroxy)propyl)-1,4,7,
10-tetraazocyclodecane
1,4,7-triazacyclononane-N,N′,N″-triacetic acid (NOTA)
1,4,8,11-tetraazacyclotetradecane-N,N′,N″,N′″-tetra-acetic
acid (TETA)
diethylenetriaminepenta-acetic anhydride or 1,4,7-
triazaheptane-N,N′,N″-pentaacetic acid (DTPA)
ethylenediamine tetra-acetic acid (EDTA)
deferoxamine (DFA)
6-histidines (6-His, or His-His-His-His-His-His)
oxalate
ethylenediamine
o-phenanthroline or 1,10-phenanthroline
dipyridyl or 2,2′-bipyridine
diethylenetriamine
8-hydroxyquinoline
dimethylglyoxime
sulfur-nitrogen chelators (e.g., bis-
thiosemicarbazone)

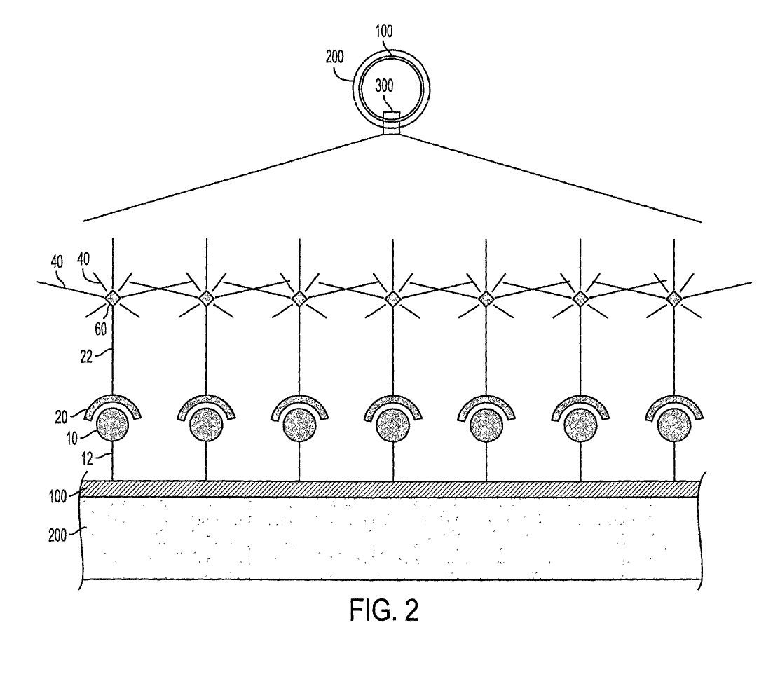

According to an alternative embodiment of the present invention the restenosis disease process is inhibited by particles and/or radiation produced as a result of neutron capture. Referring to FIG. 2, radioactive moiety 30 is replaced by neutron-capture moiety 60 attached directly to, or via linker 22 to the second member 20 of a specific binding pair.

As appreciated by the skilled artisan, neutrons can interact with the nucleus of atoms in different ways, resulting in the emission of one or more types of particles and/or electromagnetic radiation of different energies. The particles and/or radiation produced are a function of the energy of the neutron that interacts with the nucleus, the mode of interaction, and the atomic structure of the nucleus.

Neutron-capture moiety 60 consists of, or comprises an atom or ion capable of emission of a desirable particle and/or electromagnetic radiation after absorbing a neutron, according to the requirements of the skilled artisan. Such an atom or ion is referred to herein as a neutron-capture agent.

After the stent of FIG. 2 is implanted into a patient, a neutron-capture moiety 60 attached to the second member 20 of a specific binding pair is immobilized to the stent according to the methods of the instant invention. Thereafter, the artery of the patient containing the stent is exposed to a neutron flux that typically, but not necessarily, originates outside of the patient's body. Possible sources include nuclear reactors, and particle accelerators. Neutrons useful for the purpose of the present invention include but are not limited to thermal neutrons and epithermal neutrons.

After a neutron of the appropriate energy is absorbed (captured) by the neutron-capture agent contained in the neutron-capture moiety, particles and/or radiation is emitted as the nucleus returns to a less energetic state. The emitted particles and/or radiation is then absorbed by the cells of the artery surrounding or proximal to the stent, the growth of which is thereby inhibited.

According to yet another embodiment, the neutron-capture agent is also a radioactive atom or ion, such that the restenosis disease process can be inhibited both by the products of radioactive decay, and by particles and/or radiation produced after neutron-capture.

Methods for producing neutron-capture moiety 60 are well known to the skilled artisan, as are methods for attaching, covalently or non-covalently, the moiety to second member 20 of a specific binding pair, directly, or through linker 22. In many circumstances, the methods for producing and attaching neutron-capture moiety 60 are similar to those for producing and attaching radioactive moiety 30. For example, if it is desirable that the neutron-capture agent be a metal atom or ion, then neutron-capture moiety 60 can comprise the atom or ion complexed with a metal chelating moiety. Other methods for producing a neutron-capture moiety are known in the art.

For every element and isotope thereof, there exists a distinct probability of interaction between a nucleus and a neutron of a given energy. This probability of interaction is named the neutron capture cross-section of the isotope, and is measured in units of barns.

Frequently, it will be desirable for the neutron capture agent contained in the neutron-capture moiety 60 to be an atom with a large neutron-capture cross section, particularly for thermal or epithermal neutrons. In this manner, the therapeutic effectiveness of a relatively low energy neutron flux beam can be maximized.

Neutron-capture cross sections for use in the present invention may usefully be as low as about 100,000 barns, 10,000 barns, 1,000 barns, 100 barns, 10 barns, 1 barn, or 0.1 barns. Neutron capture cross sections for use in the present invention may usefully be as high as about 1 barns, 10 barns, 100 barns, 1,000 barns, 10,000 barns, 100,000 barns, or 500,000 barns.

Examples of elements useful as neutron-capture agents include, but are not limited to the following: Ac, B, Cd (including 113Cd), Dy (including 164Dy), Er, Eu (including 151Eu), Gd, Au, Hf, In, Ir, Hg, Ho (including 165Ho), Pu, Pa, Rh, Sm (including 149Sm, 152Sm), and Tm. Isotopes of gadolinium (Gd) useful for purposes of the instant invention include 152Gd, 153Gd, 155Gd, and 157Gd. Other useful isotopes of gadolinium and the other exemplary elements are known to the skilled artisan.

According to an alternative embodiment of the stent and methods of the instant invention, the immobilized molecular entity itself comprises a chelating moiety capable of chelating a radioactive or neutron capture agent. Rather than binding a second molecular entity to the immobilized entity, which second entity is complexed with a radioactive or neutron capture moiety, a radioactive or neutron capture agent is made to contact the immobilized molecular entity according to the methods of the instant invention. In this manner, the immobilized entity chelates the radioactive or neutron capture agent directly and the stent is thereby rendered radioactive or responsive to a neutron flux without the participation of an intermediary molecule.

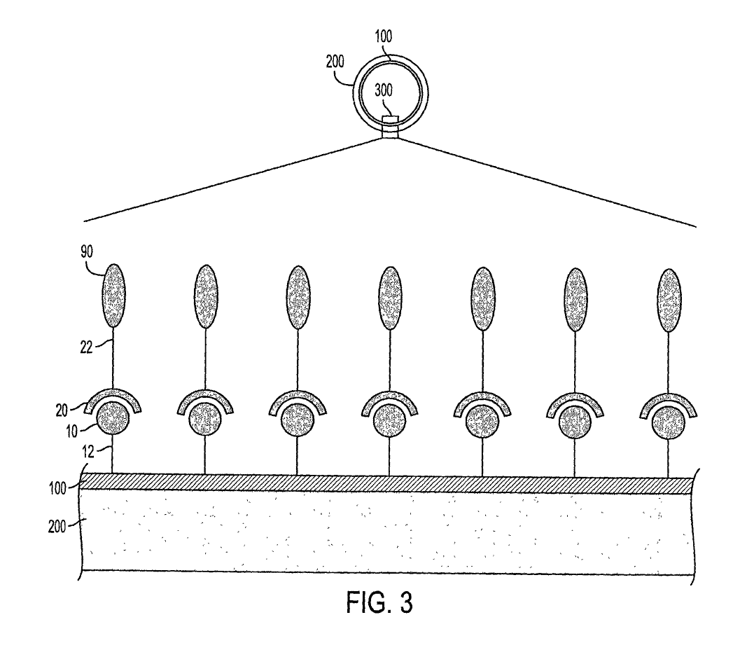

FIG. 3 schematically shows an alternative embodiment of the present invention, according to which radioactive moiety 30, or neutron capture moiety 60 is replaced by a moiety 90 consisting of or comprising a molecular entity capable of inhibiting restenosis, also called a restenosis inhibitory moiety or agent. Moiety 90 is attached directly to second member 20, or indirectly through a linker 22, and may be attached covalently or noncovalently. According to an alternative embodiment, restenosis inhibitory moiety 90 itself serves as the second member of a specific binding pair capable of binding directly to the first member. According to yet further embodiments, a restenosis inhibitory agent can be bound directly, covalently or noncovalently, to the stent surface, the surface of a coating material, or incorporated or otherwise introduced into a coating material.

For purposes of the present invention, moieties capable of inhibiting unwanted cellular growth includes, inter alia, moieties capable of inhibiting restenosis. Furthermore, moieties capable of inhibiting restenosis includes, inter alia, radioactive moieties, neutron-capture moieties and restenosis inhibitory moieties.

A restenosis inhibitory agent or moiety is a molecular entity (i.e., nucleus, atom, ion, molecule, compound, substance, or drug) capable of inhibiting restenosis by a mechanism, even if unknown, distinct from that of emission of radioactivity. Although not wishing to be bound by theory, restenosis inhibitory moiety 90 can effect restenosis inhibition by a variety of mechanisms known to the skilled artisan, although knowledge of such mechanisms is not considered necessary. Identification of a restenosis inhibitory agent can be determined empirically.

Restenosis inhibitory moieties includes growth or proliferation inhibitory moieties that inhibit growth or proliferation of various cell types involved in restenosis, in particular arterial smooth muscle cells. Growth or proliferation inhibitory moieties, radioactive moieties, and neutron-capture moieties are also effective for inhibiting unwanted cellular growth distinct from restenosis, e.g., tumorigenesis.

For purposes of the present invention, “proliferation” refers to the process of cellular division, either mitosis or meiosis, whereby increased cell numbers result. The term “growth” refers both to increase in cell mass or size, as well as cell physiological processes necessary to support a cell's life. Unless otherwise indicated, the phrase “growth inhibitory moiety” includes moieties effective both for inhibiting cellular growth and inhibiting cellular proliferation. The phrase “growth inhibitory moiety” is synonymous with the phrase “growth inhibitory agent.”

Growth or proliferation inhibition can occur by specific activation of cell surface receptors that convey an inhibitory intracellular signal. Such receptors include those that specifically bind and respond to inhibitory growth factors (e.g., TGF-β1) and inhibitory cytokines. Other growth or proliferation inhibitors require access to the cell interior where they interfere with the function of one or more molecules necessary for growth or proliferation.

Examples of mechanisms by which growth or proliferation inhibitory agents function include but are not limited to interference with DNA replication, e.g., by agents that inhibit topoisomerase I and II, or intercalate into DNA; interference with mitosis, e.g., by agents that stabilize microtubules, or inhibit mitotic spindle formation; interference with DNA, RNA or protein synthesis, e.g., by agents that inhibit DNA or RNA polymerase, or inhibit ribosome function; interference with intracellular or extracellular transport; interference with protein degradation; modulation of gene regulation; interference with intracellular signaling, e.g., by inhibiting hormone secretion, or receiving and correctly interpreting signals received from other cells; activation of apoptosis or programmed cell death. Other growth or proliferation inhibitory agents are within the knowledge of the skilled artisan.

Because restenosis is a complex process involving many cellular activities in addition to growth or proliferation, restenosis inhibitory moieties also include moieties effective for inhibiting restenosis that operate by mechanisms distinct from growth or proliferation inhibition. Thus, for purposes of the present invention, the group of moieties that are “restenosis inhibitory moieties” is broader than, and encompasses moieties that are “growth inhibitory moieties.” The phrase “restenosis inhibitory moiety” is synonymous with the phrase “restenosis inhibitory agent.”

Mechanisms of restenosis inhibition include but are not limited to inhibition of migration of cells (particularly smooth muscle cells), e.g., by inhibition of extracellular matrix deposition, and inhibition of plasmin and metalloproteinases; stimulation of vasodilation, e.g., by stimulation of nitric oxide production, and inhibition of smooth muscle cell contraction; inhibition of inflammation, e.g., by inhibition of pro-inflammatory cytokines; inhibition of thrombus formation, e.g., by inhibition of platelet function including adhesion and aggregation; immunosuppression by modulating the activity of macrophages and monocytes, endothelial cells, basophils, fibroblasts and B and T lymphocytes.

Examples of molecular entities useful as growth and/or restenosis inhibitory agents for purposes of the present invention include but are not limited to the entries in the following table.

TABLE 10
Restenosis Inhibitory Agents

Nitric oxide (NO) donors, including molsidomine,
linsidomine, sodium nitroprusside, nitroglycerol.
Tetanus toxin.
Diptheria toxin.
Ricin and other lectins.
Activators of soluble guanylate cyclase, including BAY
41-2272 (5-(Cyclopropyl-2-[1-(2-fluoro-benzyl)-1H-
pyrazolo[3,4-n]pyridin-3-yl]-pyrimidin-4-ylamine).
Smooth muscle cell relaxants, including hydralazine.
Ca²⁺-channel blockers, including verapamil, diltiazem,
nifedipine and nimodipine.
Angiotensin converting enzyme (ACE) inhibitors,
including captopril, enalapril, lisinopril and
quinapril.
Angiotensin II receptor antagonists, including
losartan, candesartan, irbesartan and valsartan.
Immunosuppressants, including dexamethasone,
betamethasone, prednisone and other corticosteriods
and steroids.
Smooth muscle cell migration and growth inhibitors,
including 17-beta-estradiol.
T-cell inhibitors, including FK506 (Tacrolimus) and
cyclosporine.
B-cell inhibitors, including mycophenolic acid.
VEGF, VEGF-receptor activators.
Tranilast.
Cyclooxygenase-2 (COX-2) antagonists, including
meloxicam, celebrex and vioxx.
Cyclooxygenase-1 (COX-1) antagonists, including
aspirin, indomethacin, diclofenac, ibuprofen and
naproxen.
Pro-urokinase inhibitors, including plasminogen
activator inhibitor-1 and other serpins.
Thrombin inhibitors, including hirudin, hirulog,
agratroban and PPACK.
Monocyte inhibitors, including interleukin-10 (IL-10).
Sirolimus, SDZ RAD (40-O-(2-hydroxyethyl)-rapamycin
and other rapamycin derivatives.
PDGF-antagonists.
Mitosis inhibitors, including paclitaxel, vinblastin,
cholchicine and combretastatin A4.
DNA replication inhibitors, including cisplatin,
mitoxantrone and topotecan.
Dihyrofolate reductase inhibitors, including
methotrexate.
Cell cycle kinase inhibitors, including flavopiridol.
Anti-cancer chemotherapeutic agents.
Ticlopidine.
Anticoagulants.
Fish oils.
Thromboxane antagonists.
Statins.
Serotonin antagonists.
Integrin IIb/IIIa inhibitors.
Probucol.
Trapidil.
Cilostazol.
Angiopeptin.
Factor Xa inhibitor.
Tissue factor-factor VIIa complex inhibitor.

As shown in FIG. 3 restenosis inhibitory agents may be stably attached or immobilized to the stent surface or stent coating. This arrangement will be typical when it is desirable to inhibit restenosis in the immediate vicinity of the stent, and/or to inhibit the growth of cells on the stent.

According to an alternative embodiment, restenosis inhibitory agents can be immobilized on the stent or coating in a manner that allows the agent to be released from the stent. This arrangement will be advantageous if the mechanism of action of the agent requires that it enter the interior of a cell to be effective, and/or if is desirable that the agent be effective against cells not in the immediate vicinity of the stent, i.e., the agent can be released from the stent surface and diffuse to cells to have its inhibitory effect.

Release of the restenosis inhibitory agent from the stent can be accomplished in different ways according to the knowledge of the skilled artisan. For example, linker 22 can comprise a protease recognition site that is cleaved by a protease displayed on a cell surface, or secreted by a cell. Alternatively, a restenosis inhibitory agent can be immobilized in the form of a pro-agent that is modified and released by cellular activity.

According to an alternative embodiment, a restenosis inhibitory agent can be incorporated in a stent coating in a manner that allows that agent to diffuse out of the coating at a known rate (hours, days or weeks) to interact with cells not in immediate contact with the stent, and thereby disrupt cellular activities leading to restenosis. Methods for incorporating a restenosis inhibitory agent into a coating, and methods for controlling dosage and the rate of release are within the knowledge of the skilled artisan. Diffusion of inhibitory agents can be accelerated, for example, by making the coating material biodegradable or dissolvable.

The coating into which the agent is incorporated can be a polymer coating, ceramic coating, or other coating materials known to the skilled artisan. If a coating is not to biodegrade or dissolve while the stent is implanted in a patient, the coating will typically be highly porous such that there is a large surface area for restenosis inhibitory agents to adsorb to. Prior to implantation of a coated stent, a predetermined amount of restenosis inhibitory agent can be preloaded into the porous coating by dipping the stent into, or spraying with a solution of the agent dissolved in an aqueous or organic solvent, followed by removal of excess agent.

An example of a biostable, non-dissolvable polymer coating that can be made porous is polytetrafluoroethylene (PTFE or Teflon®), and an example of a biostable, non-dissolvable ceramic coating that can be made porous is aluminum oxide (AlO₂). Other polymeric and ceramic materials suitable for purposes of the present invention are within the knowledge of the skilled artisan.

According to an alternative embodiment, restenosis inhibitory agents can be mixed, at a predetermined concentration, into the polymerization solution used to create the polymer coating. This approach is more typically used in the case of biodegradable or dissolvable polymer coatings.

Restenosis inhibitory agents can also be loaded into a stent coating according to the localized administration methods disclosed herein. This approach is desirable, for example, to recharge with restenosis inhibitory agent the coating of a stent that has been depleted through continuous diffusion of the agent out of the coating, or to charge the coating with a new type of restenosis inhibitory agent.

Restenosis inhibitory agents can also be combined with radioactive and/or neutron capture moieties to create a system for inhibiting restenosis by different mechanisms simultaneously. For example, diffusable restenosis inhibitory agents can be incorporated in a polymer or ceramic coating of the stent, upon which coating are also stably immobilized radioactive moieties. In this manner, radioactivity inhibits cellular proliferation and the restenosis inhibitory agent also can inhibit restenosis by interfering with cellular proliferation or by entirely different mechanism.

According to an alternative embodiment, radioactive moieties are stably immobilized to the stent surface, and diffusable restenosis inhibitory agents are incorporated in a polymer or other coating applied thereto. Other arrangements will be apparent to the skilled artisan in light of the teachings of this disclosure.

Stents of the present invention are implanted in patients in need of therapy for restenosis according to methods well known in the art. However, such methods of are not limiting and the methods of the instant invention are fully compatible with methods of stent implantation yet to be developed.

A stent of the present invention may be implanted after an atherosclerotic plaque has been treated by balloon angioplasty (also called percutaneous transluminal angioplasty (PTCA)); alternatively, a stent may be implanted simultaneous with the therapeutic compression of a plaque lesion, also usually by balloon angioplasty. Stents and methods of the present invention may also be beneficially employed in other circumstances, e.g., after atherectomy, where it is desirable to inhibit restenosis or related disease processes typified by unwanted cellular growth.

A typical method for implantation is to attach a stent, upon which is immobilized a first member of a specific binding pair, to a balloon catheter device in a nonpermanent fashion, e.g., by crimping. According to methods well known in the art, the catheter-stent combination is inserted into an artery of the patient, usually somewhat distal from the artery to be treated. With the assistance of fluoroscopy the catheter is advanced through the arterial system until the stent is located at the site of the lesion to be treated. After the stent is properly placed the balloon is expanded, thereby expanding the stent. After the stent has been sufficiently expanded the inflating balloon is deflated and the catheter removed.

According to an alternative embodiment, after balloon angioplasty, atherectomy, or other technique has been used to treat a plaque lesion, a self-expanding stent, to which is also immobilized a first member of a specific binding pair, is implanted into a patient according to methods well known in the art.

Upon successful implantation of a stent according to the instant invention, the stent is rendered capable of inhibiting unwanted cellular growth by administering locally a moiety capable of inhibiting unwanted cellular growth. According to a preferred embodiment, the moiety comprises a radioactive second member of a specific binding pair capable of specific binding with a first member of a specific binding pair immobilized on the stent, or alternatively, a coating of the stent. In this manner, the stent is rendered radioactive so as to be fully effective in inhibiting restenosis.

As discussed in the background section, systemic administration of a radioactive moiety for purposes of making a medical device (e.g., a stent) capable of inhibiting unwanted cellular growth (e.g., restenosis), is expected to result in excessive exposure to radioactivity. Alternatively, if the moiety is not radioactive, but is capable of inhibiting unwanted cellular growth by a different mechanism, then systemic administration may result in excessive exposure to powerful, potentially harmful drugs. In contrast, localized administration according to the methods of the present invention eliminates, or substantially minimizes risks associated with making a medical device capable of inhibiting unwanted cellular growth.

Methods of systemic and localized administration (e.g., intravenous, intra-arterial, etc.) are within the knowledge of the skilled artisan.

A method of the present invention comprises non-systemic administration of a moiety capable both of inhibiting unwanted cellular growth and binding a medical device, and an apparatus of the present invention comprises medical devices capable of binding growth inhibitory moieties. Although the specification for the most part describes the invention in terms of several preferred embodiments, e.g., a stent comprising a first member of a specific binding pair, and a method of making the stent radioactive by localized administration of a radioactive second member of the specific binding pair, these embodiments are illustrative and not limiting.

Localized administration of a moiety for purposes of the present invention is administration that is non-systemic, and is distinguishable from systemic administration in several ways.

By systemic administration a solution containing a moiety (e.g., a radioactive second member of a specific binding pair) capable of binding a medical device (e.g., a stent comprising a first member of the specific binding pair) is administered to a patient in a manner (e.g., by intravenous injection) that permits the solution to mix with, and be diluted by, a substantial portion of the patient's blood or other bodily fluid.

In contrast, localized administration of a solution containing a moiety (e.g., a radioactive second member of a specific binding pair) capable of binding a medical device (e.g., a stent comprising a first member of the specific binding pair) is performed in a manner that avoids any substantial mixture and dilution of the solution with the patient's blood or other bodily fluid, and yet renders the medical device capable of inhibiting unwanted cellular growth (e.g., restenosis).

Minimization of mixture and dilution of the binding solution in the patient's blood offers several advantages. First, systemic administration of a given quantity of a radioactive moiety renders substantially the entire blood supply radioactive. In contrast, localized administration, properly performed, results in minimal contamination of the patient's blood, or other bodily fluid, with the radioactive moiety, or other type of moiety.

Localized administration can be distinguished quantitatively as well as qualitatively from systemic administration of a moiety. Thus, comparing administration of the same amount of a moiety administered systemically versus locally to the same test subject (at different times), or similar test subjects, the quantity of the moiety in a sample of bodily fluid (e.g. blood) taken within a given time after systemic administration will be greater by a factor of at least about 2; 5; 10; 50; 100; 500; 1,000; 5,000; 10,000; 50,000; 100,000; 500,000; 1,000,000; 5,000,000; 10,000,000, or more, as compared to the quantity of the moiety in the same volume sample taken within the same amount of time after localized administration.

As a further consequence of avoiding substantial dilution of the moiety with the blood supply (or other bodily fluid) by using localized administration, the time necessary to effect binding of the moiety to a medical device is reduced. For example, the time necessary to effect saturable binding of a radioactive second member of a specific binding pair (or other type of growth inhibitory moiety) to a stent (or other medical device) implanted in a patient is expected to be reduced by a factor of at least about 2; 5; 10; 50; 100; 500; 1,000; 5,000; 10,000, or more, as compared to when the same amount of radioactive moiety (or other type of moiety) is administered systemically.

Exemplary methods for making a stent radioactive by localized administration of a radioactive second member of specific binding pair are described hereafter. However, the following examples should not be construed as limiting in any way the scope of the instant invention.

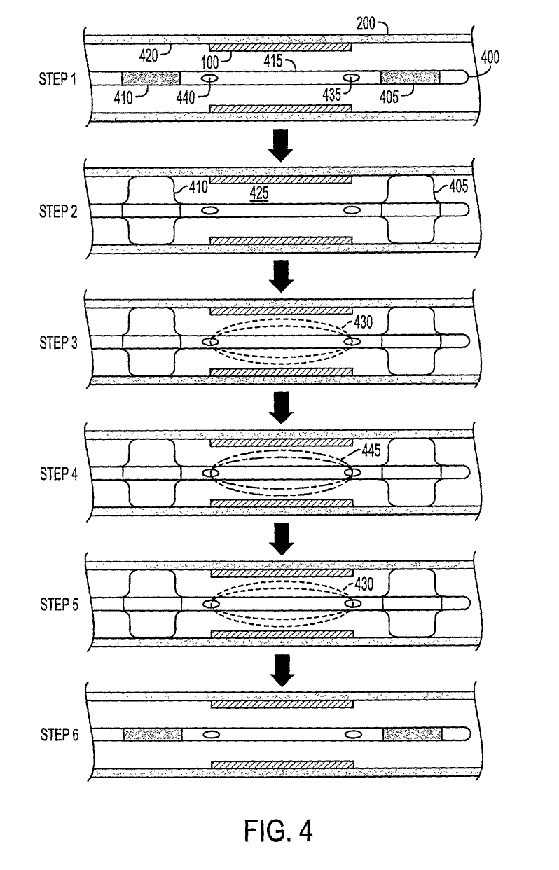

An embodiment of the methods of the present invention is illustrated in FIG. 4. After a stent 100 has been implanted, as described above, the implantation catheter is removed from the patient, and a delivery catheter 400 is introduced into and advanced through the patient's arterial system. The catheter has at least two balloons, one distal 405 and one proximal 410 to the site where the catheter is introduced into the patient's body.

According to the knowledge of the skilled artisan, the delivery catheter may be a perfusion catheter designed in such a way that when both balloons are inflated some blood flow is preserved across the arterial region temporarily occluded by inflation of the balloons (not shown). In this manner tissues downstream of the expanded balloons are less likely to become ischemic.

Between the balloons is a portion 415 of the catheter that is substantially nonexpansible. Different catheters can be designed and produced wherein the length of the nonexpansible portion is greater or longer to match the length of different stents useful for implanting into patients. Usually, the length of the nonexpansible portion is somewhat greater than the stent it is designed to be used with.

Initially, both balloons are deflated as the catheter is advanced to the site where the stent 100 was prior implanted. The catheter is further advanced through the expanded stent until such point that the distal balloon has completely cleared the distal end of the stent, and the proximal balloon is clear of the proximal end of the stent; i.e., both ends of the stent 100 surround the nonexpansible portion of the catheter (shown in STEP 1). Such positioning is usually accomplished using fluoroscopy, aided by the presence of radio-opaque markers within or at one or more of the edges of both balloons (not shown).

When the catheter is properly placed, the balloons are inflated simultaneously or one at a time in either order (shown in STEP 2). Usually, the balloons of the catheter are not inflated to the same extent or to the same pressure as the balloon used to compress a plaque lesion, or expand a stent. In this way the likelihood that the arterial walls 420 will be injured is minimized. Rather, the balloons are expanded sufficiently to create a seal such that the volume 425 formed between the expanded balloons is rendered substantially fluidly non-communicating with either the upstream or downstream volumes of blood bracketing the balloons. As a result, the expanded stent 100 is rendered essentially completely contained within this isolated volume.

The catheter is also outfitted with channels (not shown) that provide for fluid communication between the exterior of the patient's body, and the isolated volume between balloons. At minimum, the catheter possesses two such channels, an inlet channel and an outlet (or exit) channel. Through the inlet channel fluids can be delivered into the isolated volume, which fluid is drained from the volume though the outlet channel. In this manner, fluids can be delivered to the isolated volume. According to alternative embodiments, the catheter contains one or more valves (not shown) that prevent the backflow of fluids introduced into the channels.

Examples of delivery catheters are disclosed in U.S. Pat. Nos. 4,708,718; 5,213,577; 5,328,471; 5,611,775; 5,833,658, each of which is incorporated herein by reference in its entirety.

According to an embodiment of the current invention the isolated volume is washed with isotonic phosphate buffered saline, or other physiologically compatible fluid 430 (washing solution), so as to remove substantially all the arterial blood trapped between the expanded balloons, and to wash the stent, catheter and arterial walls (shown in STEP 3).

Washing solution 430 is delivered under pressure from a reservoir (not shown), through the inlet channel, debouching into the volume through a delivery inlet port 435. The fluid thereby delivered is then allowed to drain through a delivery outlet port 440 through the exit channel and ultimately to a collecting reservoir (not shown) that is typically equilibrated to atmospheric pressure. The pressure differential between the inlet and outlet is sufficient to fill and drain the volume in reasonable time, but not so great as to generate shear forces in the fluid within the volume so strong as to damage the endothelial cell lining of artery walls 420.

Although inlet port 435 is shown as being close to distal balloon 405, and outlet port 440 is shown as being close to proximal balloon 410, alternative embodiments are possible wherein their respective positions are reversed. If so reversed, the flow of fluid illustrated in FIG. 4 would also be reversed. The orientation and position of the inlet and outlet ports may be varied according to the knowledge and requirements of the skilled artisan.

Thereafter, a physiologically compatible fluid 445 (binding solution), in which is dissolved or suspended a second member of a specific binding pair complexed with a radioactive or nuclear capture moiety, is delivered into the volume between the balloons (shown in STEP 4). If, as discussed above, the stent of the present invention has immobilized thereto a plurality of different first members of specific binding pairs, the skilled artisan may optionally include in the binding solution second members complementary to one or more of the different first members.

Usually, if multiple specific binding pairs are used, they are chosen such that there is substantially no cross-reactivity among them. That is, no second member of the specific binding pair will bind a first member other than the first member to which it is complementary, and vice versa. However, as appreciated by the skilled artisan, there may be occasions where it is desirable that the second member of the specific binding pair binds specifically to more than one first member.

The solvent of the washing and binding solutions is usually water, but other fluids suitable for purposes of the present invention will be apparent to the skilled artisan, e.g., chlorofluorocarbons, and other agents that may find use as blood substitutes.

In all circumstances the washing and binding solutions are physiologically compatible. As a result, one or more characteristics of the solutions will be similar to blood, including but not limited to tonicity, osmoticity, osmolarity, osmolality, and pH. In addition to solvent, the washing and binding solutions may comprise salts, sugars, pH buffers, macromolecules and other components, such as blood anticlotting agents (e.g., heparin) according to the knowledge and requirements of the skilled artisan.

All components of the solutions must also be compatible with the function of both the first and second members of the specific binding pair, and not interfere substantially with their binding. In particular for the binding solution, it may usefully include agents that promote the stability of the second member of the specific binding pair, especially when said second member comprises a large biomolecule such as an antibody or other large protein. One example of a stabilizing agent is human serum albumin (HSA), and other such useful agents will be apparent to the skilled artisan.

The composition of the washing solution and the binding solution may be similar or different, depending on the requirements of the skilled artisan. Information useful to the skilled artisan regarding the composition of the washing and binding solutions may be found in “Remington: The Science and Practice of Pharmacy,” 20^{th} Edition, A. R. Gennaro, Editor, Lippincott Williams & Wilkins, Baltimore, Md., 2000, which is incorporated herein by reference in its entirety.

Usually, in the course of making an implanted stent radioactive the binding solution is delivered from a separate reservoir (not shown) than the washing solution. According to the requirements of the skilled artisan, both solutions may be prewarmed to body temperature and/or oxygenated prior to use.

Care must be taken to calculate the appropriate volume of binding solution to be delivered from the reservoir into the catheter and thereafter into the volume between the balloons. The volume of the channel in the catheter leading from the reservoir to the inlet port represents a void volume that must be filled before any fluid can be delivered into the volume between the balloons. Thus, after washing the stent and artery, the void volume must first be filled with binding solution before any such solution can be delivered from the reservoir into the volume formed by the balloons. Thereafter, sufficient binding solution 445 is delivered from the reservoir such that a substantial proportion of the washing solution is expelled from the volume between the balloons into the outlet port 440 and exit channel.

According to an embodiment of the present invention, the binding solution is circulated around the stent to ensure that substantially all the stent surfaces bearing the immobilized first member of the specific binding pair are contacted with binding solution at full concentration. Different methods for effecting circulation will be apparent to the skilled artisan.

One method for circulation (not shown) is to deliver binding solution until a predetermined volume enters the outlet channel. Pressure is then alternately applied to the fluid in the outlet channel and then to the fluid in the inlet channel in one or more cycles, the total number of which is to be determined by the skilled artisan. When pressure is applied to one channel, less pressure is applied to the other channel. In each cycle, fluid enters and exits the volume between the balloons and in this manner circulates, mixing the contents. During such cycling, care is taken that any washing solution in the void volume of the outlet channel is not reintroduced into the volume formed by the balloons. In this manner the binding solution is not diluted by the washing solution.

According to an alternative embodiment, the cycle comprises a cycle of positive and negative, or negative and positive, pressure applied to one channel.

After the binding solution 445 is delivered into the volume surrounding the stent between the balloons, the solution is allowed to contact the stent for sufficient time for the second member of the specific binding pair complexed with the to contact and bind the first member of the specific binding pair immobilized to the stent.

The binding reaction is typically allowed to progress until substantially all first members of the specific binding pair immobilized to the stent that are functional are bound by second members of the binding pair, i.e., until such time that the first members are substantially saturated. According to the needs of the skilled artisan, nonsaturating binding of the first member by the second of the specific binding pair is also possible. However, saturating binding is frequently desirable because it facilitates control over the number of radioactive moieties indirectly bound to the stent.

The time and conditions necessary to effect saturable binding can be determined empirically or theoretically according to the knowledge of the skilled artisan. Factors that may be considered include but are not limited to the number of functional first members immobilized to the stent; the number of binding sites for the second member per first member; concentration of second member in the binding solution; the number of binding sites for the first member per second member; temperature; concentration of salts, buffers and other constituents of the binding solution; viscosity of the binding solution; pH of the binding solution; concentration of nonspecific binding sites of the second member; and the binding rate constants, including both kₒₙ and k_{off}. In many circumstances the most important factor is likely to be the concentration of the second member of the specific binding pair in the binding solution.

Additional information regarding receptor-ligand binding can be found in “Current Protocols in Pharmacology,” S. J. Enna, et al., Editors, John Wiley & Sons, Inc., New York, N.Y., 2001, which is incorporated herein by reference in its entirety.

Factors influencing saturable binding can be adjusted according to the knowledge and requirements of the skilled artisan to ensure that such binding occurs within a time span consistent with minimizing risk to the patient. The time for binding can be as short as about 0.01 secs, 0.1 secs, 1 sec, 10 secs, 1min, 5 min, 10min, or 30min. In contrast, the time for binding can be as long as 1 hour, 30min, 10min, 5 min, 1min, 10 secs, 1 sec, or 0.1 secs. Other times for binding are possible according to the knowledge of the skilled artisan.

After the binding reaction is completed sufficient washing solution 430 is delivered into the catheter to displace the binding solution from the inlet channel as well as that occupying the volume surrounding the stent. Additional washing solution 430 is delivered as necessary to remove substantially all of the second member of the specific pair complexed with the radioactive moiety not bound to the first member of the specific binding pair (shown in STEP 5). In this manner the stent is rendered radioactive, and only a negligible, harmless amount of contaminating, unbound radioactive moiety is available to disperse into the patient's blood stream. Thus, according to this method of localized administration of the radioactive moiety for purposes of making the stent radioactive, deleterious side effects associated with systemic administration are minimized and possibly eliminated entirely.

During the washing phase of the method (e.g., STEP 3 and STEP 4) the radioactive binding solution and contaminated washing solution are collected and disposed of using methods appropriate for the type of radioisotope(s) used to make the stent radioactive. Such disposal methods are well known to those of skill in the relevant art.

After washing is completed, the balloons 405 and 410 are deflated (shown in STEP 6), and the catheter 400 is removed from the patient (not shown) who is thereafter monitored according to the knowledge of the skilled artisan.

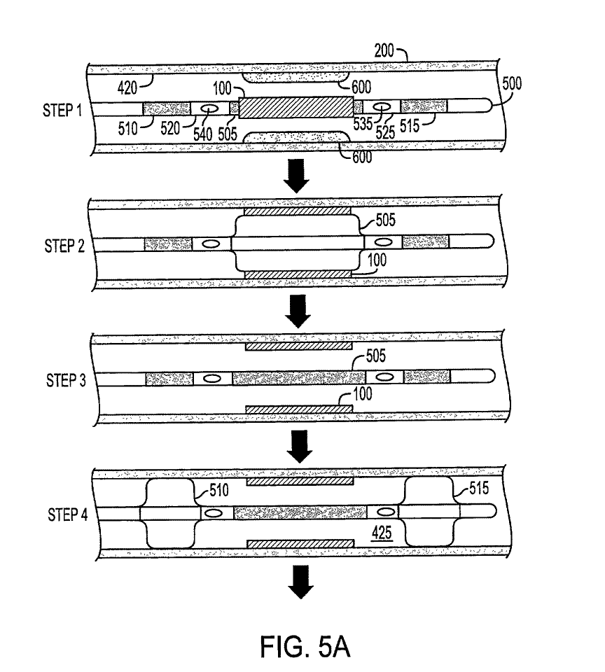

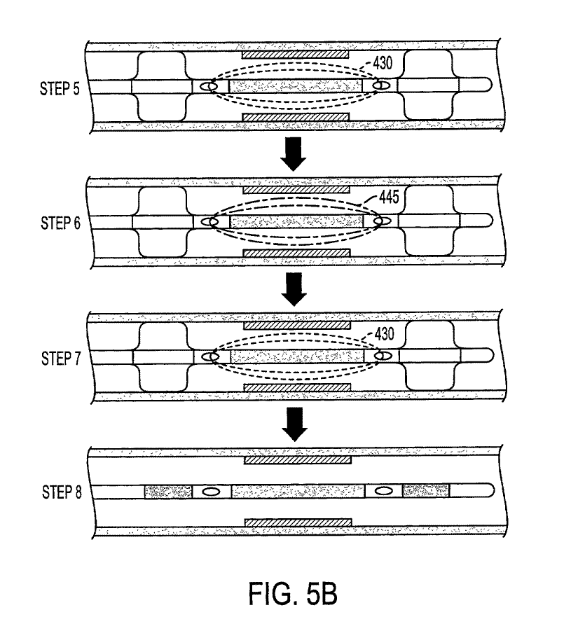

An alternative embodiment of the methods of the present invention is illustrated in FIGS. 5A and 5B, according to which the catheter used to expand the stent for implantation also serves as the delivery catheter. The catheter 500 possesses three balloons. In overall aspect of function and appearance this catheter is similar to the two-balloon catheter described above and in FIG. 4. However, in place of the substantially nonexpansible region between the balloons (e.g., 415 in FIG. 4) there exists a third balloon 505 for expanding the stent. Usually, a short nonexpansible portion of the catheter's shaft lies between the central stent-expanding balloon 505 and each of the proximal and distal end balloons, 510 and 515, respectively. Said nonexpansible portions 520 and 525 are usefully adapted to housing the inlet 535 and outlet 540 ports for the delivery channels (not shown). The central stent-expanding balloon 505 can be produced in different lengths to match the lengths of the different stents that can be implanted for purposes of the present invention.

Use of the combination expansion-delivery catheter allows the skilled artisan to implant a stent according to the present invention and, without the need for changing catheters, make the stent radioactive though the localized administration of binding solution. Briefly, a nonexpanded stent 100 is placed over the central balloon 505. The catheter 500 bearing the stent is then inserted into the patient's arterial system and advanced to the locus of the lesion 600 to be treated by stent implantation (shown in STEP 1). When placed correctly, the central balloon is inflated, thereby expanding the stent 100 (shown in STEP 2).

As appreciated by the skilled artisan, the stent may be implanted simultaneous with (as shown in FIGS. 5A and 5B) or subsequent to therapeutic compression of the atherosclerotic lesion.

Upon sufficient expansion of the stent to fix it in place within the artery the central balloon is deflated (shown in STEP 3). Then, both flanking balloons 510 and 515 are inflated to isolate stent 100 from the blood supply (shown in STEP 4), after which the washing (STEP 5), binding (STEP 6) and washing (STEP 7) steps for making the stent radioactive are performed, as described with respect to FIG. 4. The flanking balloons 510 and 515 are then deflated (STEP 8) and the catheter is removed from the patient (not shown).

According to an alternative embodiment of the combination expansion-delivery catheter (not shown), the central balloon comprises a membrane permeable to the second member of the specific binding pair complexed with a radioactive or neutron-capture moiety.

During stent implantation, the flanking balloons of the combination catheter are first inflated to isolate the arterial volume within which the stent is to be implanted. Then, the central balloon is expanded by filling it with pressurized binding solution. As the balloon expands it contacts and expands the stent of the present invention. During expansion, binding solution leaks out of the central balloon at a rate proportional to the hydrostatic pressure of the fluid, contacts the closely apposed stent surface, and thereby allowing the second member of the specific binding pair contained therein to bind the immobilized first member.

After the stent is sufficiently expanded, the central balloon is deflated, the stent and surrounding artery are washed with washing solution, and the flanking balloons are deflated, after which the catheter is removed from the patient.

According to other embodiments of the combination expansion-delivery catheter comprising a permeable balloon, the central balloon is permeable to radioactive or neutron-capture moieties, or radioactive or neutron-capture atoms or ions in solution.

Additional catheter designs useful for purposes of localized administration of a second member of a specific binding pair complexed with a radioactive or neutron-capture moiety are within the knowledge of the skilled artisan.

According to alternative embodiments of the present invention, the stent can be made radioactive by the localized administration of binding solution on more than one occasion. Repeating administration may be desirable when the radioactivity associated with the first administration has decayed but the restenosis disease process has not been fully inhibited. Other reasons for repeating the administration will be apparent according to the knowledge and requirements of the skilled artisan. Upon any or all administrations of a second member of a specific binding pair, said second member may be complexed with a neutron capture moiety in the alternative to a radioactive moiety.

As a first example, the stent of the instant invention may have immobilized thereto a plurality of different species of first members of specific binding pairs, as discussed above. According to the requirements of the skilled artisan the stent may be made radioactive on a first occasion wherein the locally administered binding solution contains a second member of a specific binding pair that specifically binds a single species of the plurality of first member species immobilized to the stent.

On a subsequent occasion the stent can again be made radioactive with the same or a different radioisotope complexed with a different second member that specifically binds a different species of first member immobilized to the stent, as compared to the second member administered on the first occasion. Additional administrations of a different second member of a specific binding pair are possible to the extent that corresponding first members are available for binding thereto. The span of time between administrations of different second members of specific binding pairs shall be determined according to the knowledge and requirements of the skilled artisan.

As a second example of making the stent radioactive on more than one occasion, the first member of the specific binding pair immobilized to the stent may comprise more than one binding site for a second member. Each such binding site is distinct from the others and binds second members of distinct specificity. Thus, during the first administration to make the stent radioactive a first radioactive second member is used. Then, during a subsequent administration to make the stent radioactive a second radioactive second member is used. Further iterations of making the stent radioactive are also possible. Each second member of distinct binding specificity for the first member used may have complexed thereto the same or a different radioisotope as used in a prior administration. A neutron capture agent may be complexed to one or more of the second members in the alternative.

As a third example of making the stent radioactive on more than one occasion a third molecular entity that recognizes and binds specifically to the second member of the specific binding pair is administered in binding solution, which third molecular entity is itself complexed to a radioactive moiety. The second member and the third molecular entity comprise a specific binding pair, and analogously, the second member of the specific binding pair functions as a first member immobilized to the stent, and the third molecular entity functions as a second member. The third molecular entity can also comprise a neutron capture moiety in the alternative to a radioactive moiety.

As a fourth example of making the stent radioactive on more than one occasion, the stent can be “recharged” with a radioactive second member of the specific binding pair. Recharging may be desirable according to the knowledge and requirements of the skilled artisan. Usually recharging is desirable if the radioactivity associated with the first administration has substantially decayed, but the restenosis disease process has not been fully inhibited. Recharging can be effected after the second member of the specific binding pair is removed, either actively or via a passive mechanism. Recharging is also possible if first members of the specific binding pair were not saturably bound during the first administration.

The second member of the specific binding pair bound during the first administration can be actively removed. According to the knowledge of the skilled artisan such removal can be accomplished in various ways. For example, a competitive inhibitor can be administered that competes with the second member for specific binding to the first member (or vice versa). Alternatively, a noncompetitive inhibitor can be administered that does not compete for binding, but rather binds to one or the other or both of the first and second members of the specific binding pair in a way that induces loss of binding affinity (e.g., by conformational change). Depending on the nature of the specific binding pair, other factors influencing binding may be modulated so as to effect loss of binding, such as a marked change in pH.

The disrupting agent, such as those exemplified herein above, is locally administered according to methods of the instant invention. Thus, a delivery catheter such as that described above can be reintroduced into a patient and advanced to the site in an artery where the stent is implanted. Once correctly positioned the flanking balloons are inflated to isolate the stent from the blood. After removing the blood from between the balloons and washing the stent, the disrupting solution is delivered, which solution may be similar in many respects to washing or binding solution, except that the former comprises a disrupting agent, whereas either of the latter do not. The disrupting agent is allowed to act on the stent for time sufficient to disrupt the binding of a substantial portion of the specific binding pairs. Thereafter, washing removes substantially all the unbound second member bound during the prior localized administration. Then, according to the methods disclosed herein, the stent is again made radioactive by delivering binding solution containing the second member of the specific binding pair complexed with a radioactive moiety.

Recharging the stent may also be desirable if the second member has been removed by a passive mechanism. For example, over time the second member may detach spontaneously from the first member and diffuse into the blood. If a sufficiently great proportion of radioactive second members of the specific binding pair debind from the stent before restenosis has been inhibited, recharging the stent by readministering binding solution in accord with the methods disclosed herein may be therapeutically necessary.

Whether recharging is effected after active or passive removal of the second member bound during a prior administration of binding solution the goal of recharging is usually to make the stent as radioactive as it had been at the time of the first administration.

Thus, the population of radioactive moieties used in recharging the stent comprises about as great or greater a proportion radioactive atoms as compared to the first administration.

According to the requirements of the skilled artisan the second member of the specific binding pair used to recharge the stent may be complexed with the same or a different radioisotope as used in the first or a prior administration. Alternatively, the second member used in the first and/or a subsequent recharging administration can be complexed with a neutron capture moiety.

According to the requirements of the skilled artisan, recharging of the stent may be effected more than one time.

For purposes of recharging the stent, second members of the specific binding pair used in subsequent administrations of binding solution are usually of the same type as used in the first or a prior iteration of making the stent radioactive. However, according to the knowledge and requirements of the skilled artisan, a different second member that is nonetheless capable of specifically recognizing the first member immobilized to the stent may be used in the alternative.

Methods of recharging stents using localized administration, as discussed above, can also be used to renew restenosis inhibitory agents, such as restenosis inhibitory moiety 90, that have been degraded, diffused out of a coating, or been removed, actively or otherwise. Such methods can be used to renew or change the identity of restenosis inhibitory agents immobilized to or incorporated in a stent or coating during a single localized administration session, or in separate serial localized administration sessions. Within any localized administration session, restenosis inhibitory moieties can be mixed with radioactive and/or neutron capture moieties such that a stent and/or coating can be simultaneously recharged with the different consitutents of the mixture.

In another aspect, the invention provides kits for rendering a medical device capable of inhibiting unwanted cellular growth, kits for treating a patient in need of therapy for restenosis, kits for making a medical device, including a stent, radioactive, and kits for making a medical device, including a stent, capable of inhibiting restenosis.

According to an embodiment of the present invention, kits can comprise a medical device and a label containing information for use of the medical device according to the methods of the instant invention (use label).

A medical device of such kits can have immobilized thereto a first member of a specific binding pair complementary to a second member. Such medical devices can be packaged in a manner that prevents substantially any degradation of the immobilized first member. For example, a medical device can be packaged in a light-excluding container, surrounded by a solution containing buffers, glycerol, proteins, reducing agents, or other stabilizing agents known to the skilled artisan. Alternatively, a medical device can be packaged dry, under vacuum, or an inert gas.

According to further embodiments of the present invention, the medical device contained in kits is a stent.

According to alternative embodiments of the present invention, a kit can comprise a use label and a moiety, an agent, or a second member of a specific binding pair according to the instant invention, including a moiety capable of inhibiting unwanted cellular growth, a moiety capable of inhibiting restenosis, a radioactive moiety, a neutron-capture moiety, or a restenosis inhibitory agent.

A moiety, an agent, or a second member of a specific binding pair provided in such a kit can be packaged as a solution, or in a desiccated form. If desiccated, the kit can also include a diluent and additional instructions for solubilizing the moiety, agent, or second member.

According to kits of the present invention, a moiety, an agent, or a second member of a specific binding pair can be packaged pre-associated with a radioactive atom or ion, or neutron capture atom or ion. Thus, according to these embodiments, if a moeity, an agent, or a second member comprises a chelating moiety, the second member is packaged with a radioactive or neutron capture agent already chelated.

Alternatively, a moiety, an agent, or a second member can be packaged in a form not pre-associated with a radioactive or neutron capture agent. According to this embodiment, the radioactive or neutron capture agent is provided in a separate container in the kit, in solution, or in a desiccated form. Alternatively, the radioactive or neutron capture agent can be provided by the end user. For example, a radioactive atom or ion can be prepared de novo in an accelerator or reactor present at a health care facility where a patient is to be treated.

If the radioactive or neutron capture agent is packaged separately, or prepared de novo, the kit can additionally contain instructions for associating, e.g., by chelation, the radioactive or neutron capture agent with a moiety, an agent, or a second member.

Containers in kits for a radioactive moiety, an agent, or a second member, or a radioactive atom or ion not yet associated with a moiety, an agent, or a second member can also be packaged with shielding to minimize exposure of people to radioactive decay products during transport, storage, or preparation.

Kits are typically provided with a sufficient quantity of a moiety, an agent, or a second member of a specific binding pair, as well as associated reagents, solutions, or supplies, to effect a single localized administration to a patient. However, kits for multiple administrations are also possible.

According to further alternative embodiments of the present invention, kits can comprise a use label and a means for localized administration of a moiety, an agent, or a second member of a specific binding pair to patients, including but not limited to a delivery catheter of any suitable design. Such kits can also optionally include volumes of washing solution and binding solution, into which a moiety, an agent, or a second member of a specific binding pair is diluted. If a kit is to contain a stent and a delivery catheter, the stent can be pre-loaded on the catheter ready for implantation of the stent.

Use labels can be compliant with drug or medical device regulatory authority regulations. Information contained therein can include, but is not limited to, instructions or directions for the method of application or use of the medical device, indications, effects, routes, methods, and frequency and duration of administration, and any relevant hazards, warnings, contraindications, side effects, and precautions under which practitioners can use the device safely and for the purpose for which it is intended, including all purposes for which it is advertised or represented.

Use labels can be applied to, or otherwise present on, a medical device itself. Use labels can also be packaged with a medical device, either as part of the packaging, or as an insert. For a second member of a specific binding pair, a use label can conveniently be affixed to a bottle or container containing the second member, and the kit can, if the label is so affixed directly to the container, exclude further packaging. The label can conveniently be abbreviated by provision of an Internet web site address (URL), at which site the user can obtain more detailed instructions, and/or provision of a telephone number, preferably a toll-free telephone number, e-mail address, or even a radio frequency at which the user can obtain more detailed instructions, either directly, or via an automatic response system.

Kits can also include combinations of a use label, a medical device, a moiety, an agent, a second member of a specific binding pair, a means for localized administration of a moiety, an agent, or a specific binding pair, or any other component described herein, or otherwise known to the skilled artisan.

The following example is to be used for illustrative purposes only and is not to be construed as in any way limiting the scope of the present invention.

### EXAMPLE 1

### Preparing a Biotin Labeled Stent

Stainless steel coronary artery stents, such as the JOSTENT® Flex stent, in their unexpanded state are prepared for biotin immobilization by first subjecting the stents to electrolysis in 5×SSC solution to form an oxide layer. Alternatively, an oxide layer is formed by immersion of the stents into a perchloric acid bath. After oxidation, the stents are rinsed in 100% ethanol and air dried in a 50° C. oven.

Thereafter, the oxidized stents are reacted with 10% 3-aminopropyltriethoxysilane (APS) in toluene. The stents are placed into a glass reaction dish, after which toluene is added to completely cover the stents, and heated to 50-60° C. on a heat plate. APS is added to a final concentration of 10%. The solution is gently mixed and allowed to continue for 30minutes. The solution is removed, and the stents are rinsed three times with large volumes of toluene, followed by rinsing three times in large volumes of 100% ethanol, followed by air drying in a 50° C. oven.

The stents are thereafter treated for 5 hours at room temperature with a solution of sulfosuccinimidyl-6-(biotinamido)hexanoate in pH 8.6 bicarbonate buffer. The stents are then rinsed extensively with sterile double distilled water, air dried, gamma irradiated for sterilization and then packaged for physician use.

All patents, patent publications, and other published references mentioned herein are hereby incorporated by reference in their entireties as if each had been individually and specifically incorporated by reference herein.

All names of elements or isotopes recited herein, unless stated in full, are expressed as the one or two letter abbreviation for the element as commonly understood by the skilled artisan.

All names of amino acid residues recited herein, unless stated in full, are expressed as the one or three letter abbreviation for the residue as commonly understood by the skilled artisan.

While preferred illustrative embodiments of the present invention are described, one skilled in the art will appreciate that the present invention can be practiced by other than the described embodiments, which are presented for purposes of illustration only and not by way of limitation. The present invention is limited only by the claims that follow.

1. A method of reducing restenosis in a patient vessel, comprising:
implanting a stent in the vessel, wherein the stent includes
a surface and a first member of a specific binding pair disposed on the surface
deploying a perfusion catheter in the vessel in a vicinity of the stent;
inflating an upstream balloon portion and a downstream balloon portion of the perfusion catheter to block vessel flow upstream and downstream of the stent; and
delivering a binding solution to the stent via the perfusion catheter, wherein the binding solution includes a restenosis-inhibiting moiety comprising a second member of the specific binding pair.

2. The method of claim 1, wherein the restenosis-inhibitory moiety comprises a radioactive moiety.

3. The method of claim 1, wherein the restenosis-inhibitory moiety comprises a neutron-capture moiety; and
the method further comprising exposing the stent to a neutron flux.

4. The method of claim 1 wherein the first and second members of the specific binding pair comprise first and second biomolecules being compatible to form a binding interaction with one another.

5. (canceled)

6. The method of claim 1 wherein the first member is immobilized directly to the stent.

7. The method of claim 1 wherein the first member is immobilized to a coating disposed on the stent.

8. (canceled)

9. (canceled)

10. The method of claim 1 further comprising repeating, at least one time, the step of delivering a binding solution to the stent via the perfusion catheter.

11. (canceled)

12. (canceled)

13. The method of claim 3 further comprising repeating, at least one time, the step of exposing the stent to a neutron flux.

14-20. (canceled)

21. The method of claim 1, further comprising delivering a wash solution to purge the region upstream and downstream of the stent prior to delivering the binding solution.

22. The method of claim 21, further comprising
delivering a second wash solution to purge the binding solution from the region upstream and downstream of the stent; and
withdrawing the perfusion catheter from the vessel.

23. A method of reducing restenosis in a patient vessel, comprising:
implanting a stent in the vessel, wherein the stent includes a surface and a first member of a specific binding pair disposed on the surface;
deploying a perfusion catheter in the vessel in a vicinity of the stent;
inflating an upstream balloon portion and a downstream balloon portion of the perfusion catheter to block vessel flow upstream and downstream of the stent;
delivering a wash solution to purge the vicinity of the stent; and
delivering a binding solution to the stent via the perfusion catheter, wherein the binding solution includes a restenosis-inhibiting moiety coupled to a second member of the specific binding pair.

24. The method of claim 23, further comprising delivering a second wash solution to purge the binding solution from the region upstream and downstream of the stent.

25. The method of claim 24, further comprising leaving the binding solution in contact with the stent for a period of time ranging from 1 second to 30minutes before delivering the second wash.

26. The method of claim 23, wherein the wash solution comprises an ionic buffered saline.

27. The method of claim 23, wherein the wash solution has at least one characteristic similar to blood.

28. The method of claim 23, wherein the restenosis-inhibitory moiety comprises at least one of a radioactive moiety or a neutron-capture moiety.

29. The method of claim 23, wherein the first and second members of the specific binding pair comprise compatible biomolecules.

30. The method of claim 23, wherein the stent includes a plurality of different first members of the specific binding pair and the binding solution includes a plurality of different second members of the specific binding pair, wherein each of the different second members of the specific binding pair is configured to bind with a selected first member of the specific binding pair and there is substantially no cross-reactivity between the different first members of the specific binding pair and the different second members of the specific binding pair.

31. A method of reducing restenosis in a patient vessel, comprising:
deploying in the vessel a catheter that includes a first balloon having a stent positioned thereon, and second and third balloons proximal and distal to the first balloon, wherein the stent includes a surface and a first member of a specific binding pair disposed on the surface;
deploying the stent by inflating the first balloon;
inflating the second and third balloons to block vessel flow upstream and downstream of the stent; and
delivering a binding solution to the stent via the catheter, wherein the binding solution includes a restenosis-inhibiting moiety coupled to a second member of the specific binding pair.

32. The method of claim 31, further comprising delivering a first wash solution via the catheter after inflating the second and third balloons to purge the region upstream and downstream of the stent.

33. The method of claim 32, further comprising delivering a second wash solution via the catheter after delivering the binding solution to the stent.

34. The method of claim 33, further comprising continuing to deliver the second wash solution until substantially no restenosis-inhibiting moiety is detectable in the wash solution.

35. The method of claim 31, wherein the binding solution is delivered to the stent via inlet and outlet ports in the catheter.

36. The method of claim 31, wherein the binding solution is delivered to the stent via the first balloon.

37. The method of claim 36, wherein the second balloon is permeable to the second member of the binding pair.
